(19) United States
(12) Patent Application Publication
(10) Pub. No.: US 2011/0086829 A1
(43) Pub. Date: Apr. 14, 2011

(54) COMPOSITIONS AND METHODS FOR TREATING OBESITY

(76) Inventors: Filiberto P. Zadini, Camarillo, CA (US); Giorgio C. Zadini, Camarillo, CA (US)

(21) Appl. No.: 12/901,413

(22) Filed: Oct. 8, 2010

Related U.S. Application Data
(60) Provisional application No. 61/278,632, filed on Oct. 9, 2009.

(51) Int. Cl. A61K 31/575; C07J 9/00; A61P 3/04

(52) U.S. Cl. 514/182; 552/553

(57) ABSTRACT

Compositions and methods for treating obesity in a subject are described. Some embodiments provide methods for administering pharmaceutical formulations including biocompatible emulsifiers in an amount effective to treat obesity. In some embodiments, pharmaceutical formulations include a combination of two or more biocompatible emulsifiers effective to treat obesity.

### RELATED APPLICATIONS

This application claims priority to U.S. Provisional Application No. 61/278,632 filed Oct. 9, 2009; the content of which is incorporated by reference herein in its entirety.

### FIELD OF THE INVENTIONS

Some embodiments described herein provide pharmaceutical formulations useful for treating obesity in a subject. Certain embodiments described herein comprise biocompatible emulsifiers, surfactants or detergents, for example, bile acids, terpenes, and saponins, in the systemic circulation in an effective amount to treat obesity.

### BACKGROUND OF THE INVENTIONS

The incidence of obesity and the related diseases are increasing throughout the entire industrialized world. The medical problems caused by overweight and obesity can be serious and often life-threatening, and include diabetes, shortness of breath, gallbladder disease, hypertension, elevated blood cholesterol levels, cancer, arthritis, other orthopedic problems, reflux esophagitis (heartburn), snoring, sleep apnea, menstrual irregularities, infertility and heart trouble. Moreover, obesity and overweight substantially increase the risk of morbidity from hypertension, dyslipidemia, type 2 diabetes, coronary heart disease, stroke, gallbladder disease, osteoarthritis and endometrial, breast, prostate, and colon cancers.

In general, available weight loss drugs have limited efficacy and some clinically significant side effects. Studies of the weight loss medications dexfenfluramine (Guy-Grand, B. et al. (1989) Lancet 2:1142-5), orlistat (Davidson, M. H. et al. (1999) JAMA 281:235-42), sibutramine (Bray, G. A. et al. (1999) Obes. Res. 7:189-98), and phentermine (Douglas, A. et al. (1983) Int. J. Obes. 7:591-5) have shown similar effectiveness. Studies for each demonstrated a weight loss of about 5% of body weight for drug compared with placebo. However, other serious considerations limit the clinical use of these drugs. Dexfenfluramine was withdrawn from the market because of suspected heart valvulopathy, orlistat is limited by GI side effects, sibutramine can cause hypertension, and phentermine has limited efficacy.

### SUMMARY OF THE INVENTIONS

Therefore, due to the important effect of obesity as a risk factor in serious and even fatal and common diseases there is still a need for pharmaceutical compounds and methods that are safe and useful in the treatment of obesity or for reducing body weight.

Some embodiments of the present invention provides a pharmaceutical formulation for treating obesity in a subject, comprising a hyodeoxycholic acid (HDCA) or a pharmaceutically acceptable salt, conjugate, hydrate, solvate, polymorph, or mixture thereof; wherein the HDCA or pharmaceutically acceptable salt, conjugate, hydrate, solvate, polymorph, or mixture thereof in the composition is in an amount effective to reduce the initial body weight by at least 5% in three months, four months, six months, eight months, or one year. In some related embodiments, the HDCA or a pharmaceutically acceptable salt, conjugate, hydrate, solvate, polymorph, or mixture thereof is in an amount effective to result in an average serum concentration of greater than 50 μM. In some embodiments the average or absolute serum or plasma concentration of greater than 50 μM is sustained over at least three months, 4 months, 6 months, 8 months, one year, or two years. In some related embodiments, the HDCA or a pharmaceutically acceptable salt, conjugate, hydrate, solvate, polymorph, or mixture thereof is in an amount effective to result in a serum concentration of from 15 μM to 1 M. In some related embodiments, the pharmaceutical formulation is a sustained release formulation and wherein the effective amount of the HDCA or a pharmaceutically acceptable salt, conjugate, hydrate, solvate, polymorph, or mixture thereof in the systemic circulation is sustained for at least about 2 hours.

Some embodiments of the present invention provides a method of treating obesity in a subject, comprising administering to the subject a pharmaceutical formulation comprising hyodeoxycholic acid (HDCA) or a pharmaceutically acceptable salt, conjugate, hydrate, solvate, polymorph, or mixture thereof; wherein the HDCA or pharmaceutically acceptable salt, conjugate, hydrate, solvate, polymorph, or mixture thereof in the formulation is in an amount effective to reduce the initial body weight by at least 5% in three months.

In some embodiments, the present invention provides a method of treating obesity in a subject, comprising administering to the subject a pharmaceutical formulation comprising hyodeoxycholic acid (HDCA) or a pharmaceutically acceptable salt, conjugate, hydrate, solvate, polymorph, or mixture thereof; wherein the HDCA or pharmaceutically acceptable salt, conjugate, hydrate, solvate, polymorph, or mixture thereof in the formulation is in an amount effective to induce a weight loss that is greater than the placebo effect and the mean formulation-associated weight loss exceeds the mean placebo weight loss by at least 5%.

In some embodiments, the present invention provides method of treating obesity in a subject, comprising administering to the subject a pharmaceutical formulation comprising hyodeoxycholic acid (HDCA) or a pharmaceutically acceptable salt, conjugate, hydrate, solvate, polymorph, or mixture thereof; wherein the HDCA or pharmaceutically acceptable salt, conjugate, hydrate, solvate, polymorph, or mixture thereof in the formulation is in an amount effective to cause the proportion of subjects who reach and maintain a loss of at least 5% of their initial body weight to be significantly greater in subjects on pharmaceutical formulation of the present invention than in those on placebo.

In some embodiment related to the above methods, the HDCA or a pharmaceutically acceptable salt, conjugate, hydrate, solvate, polymorph, or mixture thereof is in an amount effective to result in a serum concentration of greater than 50 μM. In some embodiment related to the above methods, the HDCA or a pharmaceutically acceptable salt, conjugate, hydrate, solvate, polymorph, or mixture thereof is in an amount effective to result in a serum concentration of from 15 μM to 1 M. In some embodiment related to the above methods, the HDCA or a pharmaceutically acceptable salt, conjugate, hydrate, solvate, polymorph, or mixture thereof is in an amount effective to result in a serum concentration of from 15 μM to 1 mM. In some embodiment related to the above methods, the pharmaceutical formulation is a sustained release formulation and the effective amount of the HDCA or a pharmaceutically acceptable salt, conjugate, hydrate, solvate, polymorph, or mixture thereof in the systemic circulation is sustained for at least about 2 hours. In some embodiment related to the above methods, the subject is simultaneously consuming a diet restricted in carbohydrates to less than 100 grams per day. In some embodiment related to the above methods, the subject is simultaneously consuming a diet restricted in carbohydrates to less than 60 grams per day. In some embodiment related to the above methods, the subject is simultaneously consuming a diet restricted in carbohydrates to less than 20 grams per day. In some embodiment related to the above methods, the subject is simultaneously consuming a diet restricted in carbohydrates to less than 10 grams per day.

Some embodiments of the present invention provides a pharmaceutical formulation for treating obesity in a subject, including a hyodeoxycholic acid (HDCA) or a pharmaceutically acceptable salt, conjugate, hydrate, solvate, polymorph, or mixture thereof; wherein the HDCA or pharmaceutically acceptable salt, conjugate, hydrate, solvate, polymorph, or mixture thereof is in an amount effective to reduce the subject's body weight by at least 5% within three months after an onset of administration. In some related embodiments, the HDCA or pharmaceutically acceptable salt, conjugate, hydrate, solvate, polymorph, or mixture thereof is in an amount effective to result in a serum concentration in the subject of greater than 50 μM, sustained over at least three months. In some related embodiments, the HDCA or pharmaceutically acceptable salt, conjugate, hydrate, solvate, polymorph, or mixture thereof is in an amount effective to result in a serum concentration in the subject of from about 15 μM to about 1 M. In some related embodiments, the formulation includes a sustained release formulation.

Some embodiments of the present invention provides, a method of treating obesity in a subject, comprising administering to the subject a pharmaceutical formulation including hyodeoxycholic acid (HDCA) or a pharmaceutically acceptable salt, conjugate, hydrate, solvate, polymorph, or mixture thereof in an amount effective to reduce the subject's body weight by at least 5% within three months after an onset of administration. In some other embodiments, the present invention provides a method of treating obesity, comprising administering to a subject a pharmaceutical formulation including hyodeoxycholic acid (HDCA) or a pharmaceutically acceptable salt, conjugate, hydrate, solvate, polymorph, or mixture thereof in an amount effective to induce in a first patient population a mean weight loss that exceeds, by at least 5%, a mean weight loss observed in second patient population to whom a placebo was administered. In some other embodiments, the present invention provides a method of treating obesity in a subject, comprising administering to the subject a pharmaceutical formulation including hyodeoxycholic acid (HDCA) or a pharmaceutically acceptable salt, conjugate, hydrate, solvate, polymorph, or mixture thereof in an amount effective to result in a proportion of subjects receiving the formulation who reach and maintain for at least one year a loss of at least 5% of their body weight to be significantly greater than a proportion of subjects receiving a placebo who reach and maintain for at least one year a loss of at least 5% of their body weight.

In some embodiment related to any of the above methods, the HDCA is in an amount effective to result in a serum concentration in the subject of greater than 50 μM. In some embodiment related to any of the above methods, the HDCA is in an amount effective to result in a serum concentration in the subject of from 15 μM to 1 M. In some embodiment related to any of the above methods, the HDCA is in an amount effective to result in a serum concentration in the subject of from 15 μM to 1 mM. In some embodiment related to any of the above methods, the formulation comprises a sustained release formulation. In some embodiment related to any of the above methods, during the administering, the subject consumes a diet restricted in carbohydrates to less than 40 grams per day. In some embodiment related to any of the above methods, during the administering, the subject consumes a diet restricted in carbohydrates to less than 60 grams per day. In some embodiment related to any of the above methods, during the administering, the subject consumes a diet restricted in carbohydrates to less than 20 grams per day. In some embodiment related to any of the above methods, during the administering, the subject consumes a diet restricted in carbohydrates to less than 40 grams per day.

Some embodiments of the present invention provides use of HDCA or a pharmaceutically acceptable salt, conjugate, hydrate, solvate, polymorph, or mixture thereof for preparation of a pharmaceutical formulation for treating obesity in a subject; wherein the HDCA or pharmaceutically acceptable salt, conjugate, hydrate, solvate, polymorph, or mixture thereof in the formulation is in an amount effective to reduce the initial body weight by at least 5% in three months after an onset of administration. Some embodiments of the present invention provides use of HDCA or a pharmaceutically acceptable salt, conjugate, hydrate, solvate, polymorph, or mixture thereof for preparation of a pharmaceutical formulation for treating obesity in a subject; wherein the HDCA or pharmaceutically acceptable salt, conjugate, hydrate, solvate, polymorph, or mixture thereof in the formulation is in an amount effective to induce in a first patient population a mean weight loss that exceeds, by at least 5%, a mean weight loss observed in second patient population to whom a placebo was administered. Some embodiments of the present invention provides use of HDCA or a pharmaceutically acceptable salt, conjugate, hydrate, solvate, polymorph, or mixture thereof for preparation of a pharmaceutical formulation for treating obesity in a subject; wherein the HDCA or pharmaceutically acceptable salt, conjugate, hydrate, solvate, polymorph, or mixture thereof in the formulation is in an amount effective to result in a proportion of subjects receiving the formulation who reach and maintain for at least one year a loss of at least 5% of their body weight to be significantly greater than a proportion of subjects receiving a placebo who reach and maintain for at least one year a loss of at least 5% of their body weight.

In some embodiment related to the above usages, the HDCA is in an amount effective to result in a serum concentration of greater than 50 μM. In some embodiment related to the above usages, the HDCA, the HDCA is in an amount effective to result in a serum concentration of from 15 μM to 1 M. In some embodiment related to the above usages, the HDCA, the HDCA is in an amount effective to result in a serum concentration of from 15 μM to 1 mM. In some embodiment related to the above usages, the HDCA the formulation comprises a sustained release formulation. In some embodiment related to the above usages, during the administering, the subject consumes a diet restricted in carbohydrates to less than 40 grams per day. In some embodiment related to the above usages, during the administering, the subject consumes a diet restricted in carbohydrates to less than 60 grams per day. In some embodiment related to the above usages, during the administering, the subject consumes a diet restricted in carbohydrates to less than 20 grams per day. In some embodiment related to the above usages, during the administering, the subject consumes a diet restricted in carbohydrates to less than 40 grams per day.

Some embodiments of the present invention provide a pharmaceutical compounds and compositions for treating obesity in a subject, comprising a biocompatible emulsifier in an amount effective to reduce the initial body weight by at least 5% in three months. Alternatively or in addition, the biocompatible emulsifier of the pharmaceutical formulation of the present invention is in an amount effective to induce a weight loss that is greater than the placebo effect and the mean formulation-associated weight loss exceeds the mean placebo weight loss by at least 5%. Alternatively or in addition, the biocompatible emulsifier of the pharmaceutical formulation of the present invention is in an amount effective to cause the proportion of subjects who reach and maintain a loss of at least 5% of their initial body weight to be significantly greater in subjects on pharmaceutical formulation of the present invention than in those on placebo.

In some embodiments, the biocompatible emulsifier of the pharmaceutical formulation comprises hyodeoxycholic acid (HDCA), or a pharmaceutically acceptable salt, conjugate, hydrate, solvate, or polymorph thereof; deoxycholic acid (DCA), or a pharmaceutically acceptable salt, conjugate, hydrate, solvate, or polymorph thereof; or a mixture thereof.

In some embodiments, the biocompatible emulsifier of the pharmaceutical formulation comprises D-limonene and/or an L-limonene, or a pharmaceutically acceptable salt, conjugate, hydrate, solvate, or polymorph thereof; perillic acid, such as S-perillic acid or D-perillic acid, or a pharmaceutically acceptable salt, conjugate, hydrate, solvate, or polymorph thereof; perillyl alcohol, such as 5-perillyl alcohol or D-perillyl alcohol, or a pharmaceutically acceptable salt, conjugate, hydrate, solvate, or polymorph thereof; or a mixture thereof.

In some embodiments, the biocompatible emulsifier of the pharmaceutical formulation of the present invention is in amount effective to result in a serum concentration of greater than 5 μM or from 15 μM to 1 mM of the emulsifier of greater than 5 μM or from 150 μM to 1 M. In some embodiments, the biocompatible emulsifier of the pharmaceutical formulation of the present invention is in amount effective to result in a serum concentration of the emulsifier.

In some embodiments, the biocompatible emulsifier comprises DCA or the pharmaceutically acceptable salt, conjugate, hydrate, solvate, polymorph, or mixture thereof, in an amount effective to result in a serum concentration of greater than 5 μM or in a range of from to 150 μM to 1 M of the DCA or the pharmaceutically acceptable salt, conjugate, hydrate, solvate, polymorph, or mixture thereof in the subject.

In some embodiments, the biocompatible emulsifier comprises HDCA or the pharmaceutically acceptable salt, conjugate, hydrate, solvate, polymorph, or mixture thereof, in an amount effective to result in a serum concentration of greater than 5 μM or in a range of from to 150 μM to 1 M of the HDCA or the pharmaceutically acceptable salt, conjugate, hydrate, solvate, polymorph, or mixture thereof in the subject.

In some embodiments, the biocompatible emulsifier comprises UDCA or the pharmaceutically acceptable salt, conjugate, hydrate, solvate, polymorph, or mixture thereof, in an amount effective to result in a serum concentration of greater than 5 μM or in a range of from 15 μM to 1 M of the UDCA or the pharmaceutically acceptable salt, conjugate, hydrate, solvate, polymorph, or mixture thereof in the subject.

In some embodiments, the biocompatible emulsifier comprises a terpene or a pharmaceutically acceptable salt, conjugate, hydrate, solvate, polymorph, or mixture thereof, in an amount effective to result in a serum concentration of greater than 5 μM or in a range of from 15 μM to 1 M of the terpene or the pharmaceutically acceptable salt, conjugate, hydrate, solvate, polymorph, or mixture thereof in the subject. In some embodiments, the terpene of the pharmaceutical formulation comprises D-limonene or a pharmaceutically acceptable salt, conjugate, hydrate, solvate, polymorph, or mixture thereof, in an amount effective to result in a serum concentration of greater than 5 μM or in a range of from 15 μM to 1 M of the D-limonene or the pharmaceutically acceptable salt, conjugate, hydrate, solvate, polymorph, or mixture thereof in the subject. In some embodiments, the terpene of the pharmaceutical formulation comprises S-perillic acid or a pharmaceutically acceptable salt, conjugate, hydrate, solvate, polymorph, or mixture thereof, in an amount effective to result in a serum concentration of greater than 5 μM or in a range of from 15 μM to 1 M of the S-perillic acid or the pharmaceutically acceptable salt, conjugate, hydrate, solvate, polymorph, or mixture thereof in the subject. In some embodiments, the terpene of the pharmaceutical formulation comprises S-perillyl alcohol or a pharmaceutically acceptable salt, conjugate, hydrate, solvate, polymorph, or mixture thereof, in an amount effective to result in a serum concentration of greater than 5 μM or in a range of from 15 μM to 1 M of the S-perillyl alcohol or the pharmaceutically acceptable salt, conjugate, hydrate, solvate, polymorph, or mixture thereof in the subject.

In some embodiments, pharmaceutical formulations comprising a combination of at least two biocompatible emulsifiers, terpene, saponin which are administered to a subject at doses effective to treat obesity, and the dose of each individual emulsifier in the combination can be lower than a dose that is effective to treat obesity when each emulsifier is administered alone.

In some embodiments, the administered pharmaceutical formulation comprises S-perillic acid or a pharmaceutically acceptable salt, conjugate, hydrate, solvate, polymorph, or mixture thereof, in an amount effective to result in a serum concentration of greater than 5 μM or in a range from 15 μM to 1 M of the S-perillic alcohol or the pharmaceutically acceptable salt, conjugate, hydrate, solvate, polymorph, or mixture thereof in the subject.

In some embodiments, a bile acid of the administered pharmaceutical formulation comprises HDCA, or a pharmaceutically acceptable salt, conjugate, hydrate, solvate, or polymorph thereof; deoxycholic acid (DCA), or a pharmaceutically acceptable salt, conjugate, hydrate, solvate, or polymorph thereof; or a mixture thereof.

In some embodiments, a terpene of the administered pharmaceutical formulation comprises D-limonene, or a pharmaceutically acceptable salt, conjugate, hydrate, solvate, or polymorph thereof; S-perillic acid, or a pharmaceutically acceptable salt, conjugate, hydrate, solvate, or polymorph thereof; S-perillyl alcohol, or a pharmaceutically acceptable salt, conjugate, hydrate, solvate, or polymorph thereof; or a mixture thereof.

In some embodiments, a bile acid of the administered pharmaceutical formulation comprises HDCA or a pharmaceutically acceptable salt, conjugate, hydrate, solvate, polymorph, or mixture thereof, and a terpene of the administered pharmaceutical formulation comprises D-limonene or a pharmaceutically acceptable salt, conjugate, hydrate, solvate, polymorph, or mixture thereof.

In some embodiments, a bile acid of the administered pharmaceutical formulation comprises DCA or a pharmaceutically acceptable salt, conjugate, hydrate, solvate, polymorph, or mixture thereof, and a terpene of the administered pharmaceutical formulation comprises D-limonene or a pharmaceutically acceptable salt, conjugate, hydrate, solvate, polymorph, or mixture thereof.

In some embodiments, a bile acid of the administered pharmaceutical formulation comprises HDCA or a pharmaceutically acceptable salt, conjugate, hydrate, solvate, polymorph, or mixture thereof, and a terpene of the administered pharmaceutical formulation comprises S-perillic acid or a pharmaceutically acceptable salt, conjugate, hydrate, solvate, polymorph, or mixture thereof.

In some embodiments, a bile acid of the administered pharmaceutical formulation comprises DCA or a pharmaceutically acceptable salt, conjugate, hydrate, solvate, polymorph, or mixture thereof, and a terpene of the administered pharmaceutical formulation comprises S-perillic acid or a pharmaceutically acceptable salt, conjugate, hydrate, solvate, polymorph, or mixture thereof.

In some embodiments, a bile acid of the administered pharmaceutical formulation comprises HDCA or a pharmaceutically acceptable salt, conjugate, hydrate, solvate, polymorph, or mixture thereof, and a terpene of the administered pharmaceutical formulation comprises S-perillyl alcohol or a pharmaceutically acceptable salt, conjugate, hydrate, solvate, polymorph, or mixture thereof.

In some Embodiments, a bile acid of the administered pharmaceutical formulation comprises DCA or a pharmaceutically acceptable salt, conjugate, hydrate, solvate, polymorph, or mixture thereof, and a terpene of the administered pharmaceutical formulation comprises S-perillyl alcohol or a pharmaceutically acceptable salt, conjugate, hydrate, solvate, polymorph, or mixture thereof.

In some embodiments, a terpene of the administered pharmaceutical formulation comprises D-limonene or a pharmaceutically acceptable salt, conjugate, hydrate, solvate, polymorph, or mixture thereof, and the pharmaceutical formulation is administered in a dose comprising an amount of D-limonene in a range of from 1 mg/kg/day to 20 g/kg/day.

In some embodiments, a terpene of the administered pharmaceutical formulation comprises S-perillic acid or a pharmaceutically acceptable salt, conjugate, hydrate, solvate, polymorph, or mixture thereof, and the pharmaceutical formulation is administered in a dose comprising an amount of S-perillic acid in a range of from 1 mg/kg/day to 20 g/kg/day.

In some embodiments, a terpene of the administered pharmaceutical comprises S-perillyl alcohol or a pharmaceutically acceptable salt, conjugate, hydrate, solvate, polymorph, or mixture thereof, and the pharmaceutical formulation is administered in a dose comprising an amount of S-perillyl alcohol in a range of from 1 mg/kg/day to 20 g/kg/day.

In some embodiments, the pharmaceutical formulation comprises a permeability enhancer comprising at least one of a non-ionic detergent, an ionic detergent, and a zwitterionic detergent. In some embodiments, the permeability enhancer comprises at least one of a non-ionic detergent, an ionic detergent, and a zwitterionic detergent. In some embodiments, the permeability enhancer comprises at least one of iontophoresis, electroporation, sonophoresis, thermal poration, microneedle treatment, and dermabrasion.

In some embodiments, the pharmaceutical formulation comprises a lipase. In some embodiments, the lipase comprises an cholesteryl ester hydrolase. In some embodiments, the lipase comprises an cholesterol esterase. In some embodiments, the pharmaceutical formulation further comprises at least one of a lysyl oxidase and a lysyl oxidase agonist.

In some embodiments, the pharmaceutical formulation further comprises a statin.

In some embodiments, the pharmaceutical formulation further comprises a liposome, wherein the liposome carries at least one of the bile acid or the pharmaceutically acceptable salt, conjugate, hydrate, solvate, polymorph, or mixture thereof and the terpene or the pharmaceutically acceptable salt, conjugate, hydrate, solvate, polymorph, or mixture thereof.

In some embodiments, the pharmaceutical formulation is administered intravenously.

In some embodiments, the pharmaceutical formulation is administered intra-arterially.

In some embodiments, the pharmaceutical formulation is administered orally.

In some embodiments, the pharmaceutical formulation is administered sublingually.

In some embodiments, the pharmaceutical formulation is administered transdermally.

In some embodiments, the pharmaceutical formulation is administered via an implantable device.

In some embodiments, the pharmaceutical formulation is administered by injection.

In some embodiments, the pharmaceutical formulation is administered transmucosally.

In some embodiments, the pharmaceutical formulation is a sustained release formulation. In some embodiments, the level of the biocompatible emulsifier in the systemic circulation of the subject is sustained for a period of at least two hours.

In some embodiments, the sustained levels of the emulsifier in the systemic circulation are greater than 50 μM. In some embodiments, the sustained levels of the emulsifier in the systemic circulation are in a range between about 60 μM and about 600 μM. In some embodiments, the sustained levels of the emulsifier in the systemic circulation are in a range between about 100 μM and about 300 μM.

In some embodiments, the sustained levels of the deoxycholic acid in the systemic circulation are greater than 50 μM. In some embodiments, the sustained levels of the deoxycholic acid in the systemic circulation are in a range between about 60 μM and about 600 μM. In some embodiments, the sustained levels of the deoxycholic acid in the systemic circulation are in a range between about 100 μM and about 300 μM.

In some embodiments, the biocompatible emulsifier comprises a mixture of ursodeoxycholic acid and deoxycholic acid in substantially equimolar amounts. In some embodiments, the emulsifier comprises hyodeoxycholic acid. In some embodiments, the sustained levels of the hyodeoxycholic acid in the systemic circulation are greater than about 50 μM. In some embodiments, the sustained levels of the hyodeoxycholic acid in the systemic circulation are in a range from about 60 μM to about 600 μM. In some embodiments, the sustained levels of the hyodeoxycholic acid in the systemic circulation are in a range from about 100 μM to about 300 μM.

Some embodiments of the present invention provide a method of treating obesity in a subject comprising administering to the subject a pharmaceutical formulation comprising a biocompatible emulsifier or a pharmaceutically acceptable salt, conjugate, hydrate, solvate, polymorph, or mixture thereof; wherein the biocompatible emulsifier in the formulation is in an amount effective to reduce the subject's body weight by at least 5% in three months from an onset of administration.

Alternatively or in addition, the biocompatible emulsifier is in an amount effective to induce a weight loss that is greater than the placebo effect and the mean formulation-associated weight loss exceeds the mean placebo weight loss by at least 5%. Alternatively or in addition, the biocompatible emulsifier is in an amount effective to cause the proportion of subjects who reach and maintain a loss of at least 5% of their initial body weight to be significantly greater in subjects on pharmaceutical formulation of the present invention than in those on placebo.

In some embodiments relating to the method of the present invention, the biocompatible emulsifier comprises hyodeoxycholic acid (HDCA), or a pharmaceutically acceptable salt, conjugate, hydrate, solvate, or polymorph thereof; deoxycholic acid (DCA), or a pharmaceutically acceptable salt, conjugate, hydrate, solvate, or polymorph thereof; or a mixture thereof.

In some embodiments relating to the method of the present invention, the biocompatible emulsifier comprises D-limonene and/or an L-limonene, or a pharmaceutically acceptable salt, conjugate, hydrate, solvate, or polymorph thereof; perillic acid, such as S-perillic acid or D-perillic acid, or a pharmaceutically acceptable salt, conjugate, hydrate, solvate, or polymorph thereof; perillyl alcohol, such as S-perillyl alcohol or D-perillyl alcohol, or a pharmaceutically acceptable salt, conjugate, hydrate, solvate, or polymorph thereof; or a mixture thereof.

In some embodiments relating to the method of the present invention, the biocompatible emulsifier is in amount effective to result in a sustained serum concentration of greater than 5 μM or from 15 μM to 1 M of the emulsifier.

In some embodiments relating to the method of the present invention, the biocompatible emulsifier comprises DCA or the pharmaceutically acceptable salt, conjugate, hydrate, solvate, polymorph, or mixture thereof, in an amount effective to result in a serum concentration of greater than 5 μM or in a range of from to 150 μM to 1 M of the DCA or the pharmaceutically acceptable salt, conjugate, hydrate, solvate, polymorph, or mixture thereof in the subject.

In some embodiments relating to the method of the present invention, the biocompatible emulsifier comprises HDCA or the pharmaceutically acceptable salt, conjugate, hydrate, solvate, polymorph, or mixture thereof, in an amount effective to result in a serum concentration of greater than 5 μM or in a range of from to 150 μM to 1 M of the HDCA or the pharmaceutically acceptable salt, conjugate, hydrate, solvate, polymorph, or mixture thereof in the subject.

In some embodiments relating to the method of the present invention, the biocompatible emulsifier comprises UDCA or the pharmaceutically acceptable salt, conjugate, hydrate, solvate, polymorph, or mixture thereof, in an amount effective to result in a serum concentration of greater than 5 μM or in a range of from to 150 μM to 1 M of the UDCA or the pharmaceutically acceptable salt, conjugate, hydrate, solvate, polymorph, or mixture thereof in the subject.

In some embodiments relating to the method of the present invention, the biocompatible emulsifier comprises a terpene or a pharmaceutically acceptable salt, conjugate, hydrate, solvate, polymorph, or mixture thereof, in an amount effective to result in a serum concentration of greater than 5 μM or in a range of from to 150 μM to 1 M of the terpene or the pharmaceutically acceptable salt, conjugate, hydrate, solvate, polymorph, or mixture thereof in the subject.

In some embodiments relating to the method of the present invention, biocompatible emulsifier comprises D-limonene or a pharmaceutically acceptable salt, conjugate, hydrate, solvate, polymorph, or mixture thereof, in an amount effective to result in a serum concentration of greater than 5 μM or in a range of from 15 μM to 1 M of the D-limonene or the pharmaceutically acceptable salt, conjugate, hydrate, solvate, polymorph, or mixture thereof in the subject.

In some embodiments relating to the method of the present invention, the biocompatible emulsifier comprises S-perillic acid or a pharmaceutically acceptable salt, conjugate, hydrate, solvate, polymorph, or mixture thereof, in an amount effective to result in a serum concentration of greater than 5 μM or in the subject in a range of from 15 μM to 1 M of the S-perillic acid or the pharmaceutically acceptable salt, conjugate, hydrate, solvate, polymorph, or mixture thereof in the subject.

In some embodiments relating to the method of the present invention, the biocompatible emulsifier comprises S-perillyl alcohol or a pharmaceutically acceptable salt, conjugate, hydrate, solvate, polymorph, or mixture thereof, in an amount effective to result in a serum concentration of greater than 5 μM or in a range of from 15 μM to 1 M of the S-perillyl alcohol or the pharmaceutically acceptable salt, conjugate, hydrate, solvate, polymorph, or mixture thereof in the subject.

In some embodiments relating to the method of the present invention, the administered comprises a combination of at least two biocompatible emulsifiers, terpene, saponin are administered to a subject at doses effective to treat obesity, and the dose of each individual emulsifier in the combination can be lower than a dose that is effective to treat obesity when each emulsifier is administered alone.

In some embodiments relating to the method of the present invention, the administered pharmaceutical formulation comprises S-perillic acid or a pharmaceutically acceptable salt, conjugate, hydrate, solvate, polymorph, or mixture thereof, in an amount effective to result in a serum concentration of greater than 5 μM or in a range of from 15 μM to 1 M of the S-perillic alcohol or the pharmaceutically acceptable salt, conjugate, hydrate, solvate, polymorph, or mixture thereof in the subject.

In some embodiments relating to the method of the present invention, the biocompatible emulsifier administered comprises HDCA, or a pharmaceutically acceptable salt, conjugate, hydrate, solvate, or polymorph thereof; deoxycholic acid (DCA), or a pharmaceutically acceptable salt, conjugate, hydrate, solvate, or polymorph thereof; or a mixture thereof.

In some embodiments relating to the method of the present invention, the biocompatible emulsifier administered comprises D-limonene, or a pharmaceutically acceptable salt, conjugate, hydrate, solvate, or polymorph thereof; S-perillic acid, or a pharmaceutically acceptable salt, conjugate, hydrate, solvate, or polymorph thereof; or S-perillyl alcohol, or a pharmaceutically acceptable salt, conjugate, hydrate, solvate, or polymorph thereof; or a mixture thereof.

In some embodiments relating to the method of the present invention, the biocompatible emulsifier administered comprises HDCA or a pharmaceutically acceptable salt, conjugate, hydrate, solvate, polymorph, or mixture thereof, and D-limonene or a pharmaceutically acceptable salt, conjugate, hydrate, solvate, polymorph, or mixture thereof.

In some embodiments relating to the method of the present invention, the biocompatible emulsifier administered comprises DCA or a pharmaceutically acceptable salt, conjugate, hydrate, solvate, polymorph, or mixture thereof, and D-limonene or a pharmaceutically acceptable salt, conjugate, hydrate, solvate, polymorph, or mixture thereof.

In some embodiments relating to the method of the present invention, the biocompatible emulsifier administered comprises HDCA or a pharmaceutically acceptable salt, conjugate, hydrate, solvate, polymorph, or mixture thereof, and S-perillic acid or a pharmaceutically acceptable salt, conjugate, hydrate, solvate, polymorph, or mixture thereof.

In some embodiments relating to the method of the present invention, the biocompatible emulsifier comprises DCA or a pharmaceutically acceptable salt, conjugate, hydrate, solvate, polymorph, or mixture thereof, and S-perillic acid or a pharmaceutically acceptable salt, conjugate, hydrate, solvate, polymorph, or mixture thereof.

In some embodiments relating to the method of the present invention, the biocompatible emulsifier comprises HDCA or a pharmaceutically acceptable salt, conjugate, hydrate, solvate, polymorph, or mixture thereof, and S-perillyl alcohol or a pharmaceutically acceptable salt, conjugate, hydrate, solvate, polymorph, or mixture thereof.

In some embodiments relating to the method of the present invention, the biocompatible emulsifier comprises DCA or a pharmaceutically acceptable salt, conjugate, hydrate, solvate, polymorph, or mixture thereof, and S-perillyl alcohol or a pharmaceutically acceptable salt, conjugate, hydrate, solvate, polymorph, or mixture thereof.

In some embodiments relating to the method of the present invention, the biocompatible emulsifier administered comprises D-limonene or a pharmaceutically acceptable salt, conjugate, hydrate, solvate, polymorph, or mixture thereof, and the pharmaceutical formulation is administered in a dose comprising an amount of D-limonene in a range of from 1 mg/kg/day to 20 g/kg/day.

In some embodiments relating to the method of the present invention, the biocompatible emulsifier administered comprises S-perillic acid or a pharmaceutically acceptable salt, conjugate, hydrate, solvate, polymorph, or mixture thereof, and the pharmaceutical formulation is administered in a dose comprising an amount of S-perillic acid in a range of from 1 mg/kg/day to 20 g/kg/day.

In some embodiments relating to the method of the present invention, the biocompatible emulsifier administered comprises S-perillyl alcohol or a pharmaceutically acceptable salt, conjugate, hydrate, solvate, polymorph, or mixture thereof, and the pharmaceutical formulation is administered in a dose comprising an amount of S-perillyl alcohol in a range of from 1 mg/kg/day to 20 g/kg/day.

In some embodiments relating to the method of the present invention, the pharmaceutical formulation administered comprises a permeability enhancer comprising at least one of a non-ionic detergent, an ionic detergent, and a zwitterionic detergent. In some embodiments, the permeability enhancer comprises at least one of a non-ionic detergent, an ionic detergent, and a zwitterionic detergent. In some embodiments, the permeability enhancer comprises at least one of iontophoresis, electroporation, sonophoresis, thermal poration, microneedle treatment, and dermabrasion.

In some embodiments relating to the method of the present invention, the pharmaceutical formulation administered comprises a lipase. In some embodiments, the lipase comprises an cholesteryl ester hydrolase. In some embodiments, the lipase comprises an cholesterol esterase. In some embodiments, the pharmaceutical formulation further comprises at least one of a lysyl oxidase and a lysyl oxidase agonist.

In some embodiments relating to the method of the present invention, the pharmaceutical formulation administered further comprises a statin.

In some embodiments relating to the method of the present invention, the pharmaceutical formulation administered further comprises a liposome, wherein the liposome carries at least one of the biocompatible emulsifiers or the pharmaceutically acceptable salt, conjugate, hydrate, solvate, polymorph, or mixture thereof and the terpene or the pharmaceutically acceptable salt, conjugate, hydrate, solvate, polymorph, or mixture thereof.

In some embodiments relating to the method of the present invention, the pharmaceutical formulation is administered intravenously.

In some embodiments relating to the method of the present invention, the pharmaceutical formulation is administered intra-arterially.

In some embodiments relating to the method of the present invention, the pharmaceutical formulation is administered orally.

In some embodiments relating to the method of the present invention, the pharmaceutical formulation is administered sublingually.

In some embodiments relating to the method of the present invention, the pharmaceutical formulation is administered transdermally.

In some embodiments relating to the method of the present invention, the pharmaceutical formulation is administered via an implantable device.

In some embodiments relating to the method of the present invention, the pharmaceutical formulation is administered by injection.

In some embodiments relating to the method of the present invention, the pharmaceutical formulation is administered transmucosally.

In some embodiments relating to the method of the present invention, the level of the biocompatible emulsifier in the systemic circulation of the subject is sustained for a period of at least two hours.

In some embodiments relating to the method of the present invention, the sustained levels of the emulsifier in the systemic circulation are greater than 50 μM. In some embodiments, the sustained levels of the emulsifier in the systemic circulation are in a range between about 50 μM and about 600 μM. In some embodiments, the sustained levels of the emulsifier in the systemic circulation are in a range between about 100 μM and about 300 μM.

In some embodiments relating to the method of the present invention, the sustained levels of the deoxycholic acid in the systemic circulation are greater than 50 μM. In some embodiments, the sustained levels of the deoxycholic acid in the systemic circulation are in a range between about 50 μM and about 600 μM. In some embodiments, the sustained levels of the deoxycholic acid in the systemic circulation are in a range between about 100 μM and about 300 μM.

In some embodiments relating to the method of the present invention, the biocompatible emulsifier comprises a mixture of ursodeoxycholic acid and deoxycholic acid in substantially equimolar amounts. In some embodiments, the emulsifier comprises hyodeoxycholic acid. In some embodiments, the sustained levels of the hyodeoxycholic acid in the systemic circulation are greater than about 50 μM. In some embodiments, the sustained levels of the hyodeoxycholic acid in the systemic circulation are in a range from about 50 μM to about 600 μM. In some embodiments, the sustained levels of the hyodeoxycholic acid in the systemic circulation are in a range from about 100 μM to about 300 μM.

In some embodiments relating to the method of the present invention, the active ingredient of the administered pharmaceutical formulation consists essentially of DCA or a pharmaceutically acceptable salt, conjugate, hydrate, solvate, polymorph, or mixture thereof, and D-limonene or a pharmaceutically acceptable salt, conjugate, hydrate, solvate, polymorph, or mixture thereof.

In some embodiments relating to the method of the present invention, the active ingredient of the administered pharmaceutical formulation consists essentially of DCA or a pharmaceutically acceptable salt, conjugate, hydrate, solvate, polymorph, or mixture thereof, and S-perillic acid or a pharmaceutically acceptable salt, conjugate, hydrate, solvate, polymorph, or mixture thereof.

In some embodiments relating to the method of the present invention, the active ingredient of the administered pharmaceutical formulation consists essentially of DCA or a pharmaceutically acceptable salt, conjugate, hydrate, solvate, polymorph, or mixture thereof, and S-perillyl alcohol or a pharmaceutically acceptable salt, conjugate, hydrate, solvate, polymorph, or mixture thereof.

In some embodiments relating to the method of the present invention, the active ingredient of the administered pharmaceutical formulation consists essentially of HDCA or a pharmaceutically acceptable salt, conjugate, hydrate, solvate, polymorph, or mixture thereof, and D-limonene or a pharmaceutically acceptable salt, conjugate, hydrate, solvate, polymorph, or mixture thereof.

In some embodiments relating to the method of the present invention, the active ingredient of the administered pharmaceutical formulation consists essentially of HDCA or a pharmaceutically acceptable salt, conjugate, hydrate, solvate, polymorph, or mixture thereof, and S-perillic acid or a pharmaceutically acceptable salt, conjugate, hydrate, solvate, polymorph, or mixture thereof.

In some embodiments relating to the method of the present invention, the active ingredient of the administered pharmaceutical formulation consists essentially of HDCA or a pharmaceutically acceptable salt, conjugate, hydrate, solvate, polymorph, or mixture thereof, and S-perillyl alcohol or a pharmaceutically acceptable salt, conjugate, hydrate, solvate, polymorph, or mixture thereof.

In some embodiments relating to the method of the present invention, the active ingredient of the administered pharmaceutical formulation consists essentially of D-limonene or a pharmaceutically acceptable salt, conjugate, hydrate, solvate, polymorph, or mixture thereof, and the pharmaceutical formulation comprises an amount of active ingredient effective to result in a serum concentration of active ingredient in the subject in a range of from 1 mM to 1 M.

In some embodiments relating to the method of the present invention, the active ingredient of the administered pharmaceutical formulation consists essentially of S-perillic acid or a pharmaceutically acceptable salt, conjugate, hydrate, solvate, polymorph, or mixture thereof, and the pharmaceutical formulation comprises an amount of active ingredient effective to result in a serum concentration of active ingredient in the subject in a range of from 1 mM to 1 M.

In some embodiments relating to the method of the present invention, the active ingredient of the administered pharmaceutical formulation consists essentially of S-perillyl alcohol or a pharmaceutically acceptable salt, conjugate, hydrate, solvate, polymorph, or mixture thereof, and the pharmaceutical formulation comprises an amount of active ingredient effective to result in a serum concentration of active ingredient in the subject in a range of from 1 mM to 1 M.

In some embodiments relating to the method of the present invention, the active ingredient of the administered pharmaceutical formulation consists essentially of D-limonene or a pharmaceutically acceptable salt, conjugate, hydrate, solvate, polymorph, or mixture thereof, and the pharmaceutical formulation is administered in a dose comprising an amount of active ingredient in a range of from 1 mg/kg/day to 20 g/kg/day.

In some embodiments relating to the method of the present invention, the active ingredient of the administered pharmaceutical formulation consists essentially S-perillic acid or a pharmaceutically acceptable salt, conjugate, hydrate, solvate, polymorph, or mixture thereof, and the pharmaceutical formulation is administered in a dose comprising an amount active ingredient in a range of from 1 mg/kg/day to 20 g/kg/day.

In some embodiments relating to the method of the present invention, the active ingredient of the administered pharmaceutical formulation consists essentially of S-perillyl alcohol or a pharmaceutically acceptable salt, conjugate, hydrate, solvate, polymorph, or mixture thereof, and the pharmaceutical formulation is administered in a dose comprising an amount of active ingredient in a range of from 1 mg/kg/day to 20 g/kg/day.

In some embodiments relating to the method of the present invention, the administering comprises performing at least one of iontophoresis, electroporation, sonophoresis, thermal poration, microneedle treatment, and dermabrasion.

In some embodiments, the method further comprises administering a statin either simultaneously or sequentially with the pharmaceutical formulation. In some embodiments, the pharmaceutical formulation further comprises the statin.

In some embodiments, there is provided a method of treating obesity in a subject comprising administering a pharmaceutical formulation comprising a biocomaptible emulsifier in an amount effective achieve a concentration of the emulsifier in the systemic circulation of at least 50 μM; wherein the concentration of the emulsifier in the systemic circulation is sustained for a period of at least two hours; wherein the concentration of the emulsifier is effective to result reduction of the initial body weight of the subject by at least 5% in three months. Alternatively or in addition, the biocompatible emulsifier is in an amount effective to induce a weight loss that is greater than the placebo effect and the mean formulation-associated weight loss exceeds the mean placebo weight loss by at least 5%. Alternatively or in addition, the biocompatible emulsifier is in an amount effective to cause the proportion of subjects who reach and maintain a loss of at least 5% of their initial body weight to be significantly greater in subjects on pharmaceutical formulation of the present invention than in those on placebo.

In some embodiments relating to this method, the biocompatible emulsifier comprises at least one of a bile acid, a saponin, a detergent, or pharmaceutically acceptable salts, conjugates, hydrates, solvates, polymorphs, or mixtures thereof. In some embodiments, the emulsifier comprises a bile acid, or pharmaceutically acceptable salts, conjugates, hydrates, solvates, polymorphs, or mixtures thereof.

In some embodiments relating to this method, the sustained levels of the emulsifier in the systemic circulation are greater than 50 μM. In some embodiments, the sustained levels of the emulsifier in the systemic circulation are in a range between about 50 μM and about 600 μM. In some embodiments, the sustained levels of the emulsifier in the systemic circulation are in a range between about 100 μM and about 300 μM.

In some embodiments relating to this method, the emulsifier comprises deoxycholic acid. In some embodiments, the sustained levels of the deoxycholic acid in the systemic circulation are greater than 50 μM. In some embodiments, the sustained levels of the deoxycholic acid in the systemic circulation are in a range between about 50 μM and about 600 μM. In some embodiments, the sustained levels of the deoxycholic acid in the systemic circulation are in a range between about 100 μM and about 300 μM.

In some embodiments relating to this method, the emulsifier comprises a mixture of ursodeoxycholic acid and deoxycholic acid in substantially equimolar amounts.

In some embodiments relating to this method, the emulsifier comprises hyodeoxycholic acid. In some embodiments, the sustained levels of the hyodeoxycholic acid in the systemic circulation are greater than about 50 μM. In some embodiments, the sustained levels of the hyodeoxycholic acid in the systemic circulation are in a range from about 50 μM to about 600 μM. In some embodiments, the sustained levels of the hyodeoxycholic acid in the systemic circulation are in a range from about 100 μM to about 300 μM.

In some embodiments, the method further comprises the use of a permeability enhancer. In some embodiments, the permeability enhancer comprises at least one of a non-ionic detergent, an ionic detergent, and a zwitterionic detergent. In some embodiments, the permeability enhancer comprises at least one of iontophoresis, electroporation, sonophoresis, thermal poration, microneedle treatment, and dermabrasion.

In some embodiments relating to this method, the pharmaceutical formation is administered intravenously. In some embodiments, the pharmaceutical formation is administered intra-arterially. In some embodiments, the pharmaceutical formation is administered orally. In some embodiments, the pharmaceutical formation is administered sublingually. In some embodiments, the pharmaceutical formation is administered transdermally. In some embodiments, the pharmaceutical formation is administered via an implantable device. In some embodiments, the pharmaceutical formation is administered by injection. In some embodiments, the pharmaceutical formation is administered transmucosally.

In some embodiments, the method further comprises administering a statin either simultaneously or sequentially with the pharmaceutical formulation. In some embodiments, the pharmaceutical formulation further comprises the statin.

In some embodiments, there is provided a method of treating obesity in a subject comprising administering a pharmaceutical formulation comprising a biocompatible emulsifier in an amount effective achieve a concentration of the emulsifier in the systemic circulation of at least 50 μM at five minutes after onset of administration; wherein the concentration of the emulsifier in the systemic circulation is sustained above 50 μM for a period of at least two hours; and wherein the concentration of the emulsifier is effective to treat obesity.

In some embodiments, the sustained levels of the emulsifier in the systemic circulation are greater than 50 μM. In some embodiments, the sustained levels of the emulsifier in the systemic circulation are in a range between about 50 μM and about 600 μM. In some embodiments, the sustained levels of the emulsifier in the systemic circulation are in a range between about 100 μM and about 300 μM.

### BRIEF DESCRIPTION OF THE DRAWINGS

FIG. 1 shows skin patch for systemic administration of a biocompatible pharmacological compound via a convenient route, the transdermal route.

### DETAILED DESCRIPTION OF THE INVENTIONS

The present invention is directed to a novel compositions and methods for treating obesity in a subject. In particular, the present invention provides methods which involve administering to a subject a composition comprising a biocompatible emulsifier (e.g., bile acids, terpenes, saponins or detergents) in which the biocompatible emulsifier is in an amount effective to reduce the initial body weight of the subject by at least 5% in a certain period of time, for example, in six weeks. These methods are particularly useful for the treatment of overweight and/or obesity, as well as other conditions relating to obesity (e.g., metabolic syndrome).

The present invention is based, in part, on the discovery that taking 250 mg of Hyodeoxycholate (HDCA) orally three times a day resulted in mild ketonuria (a medical condition in which ketone bodies are present in the urine) in a healthy individual who continued to consume his normal diet including carbohydrates. It is know in the art that when hyodeoxycholic acid (a hydrophilic bile acid) is taken orally, it is absorbed through the gut and reaches the liver, but then a large fraction of it, up to 70%, escapes the entero-hepatic circulation, and enters the systemic circulation. See Salvioli et al., European Journal of Clinical Investigation, Vol. 18(1), pp. 22-28 (February 1988). Therefore, the induction of ketonuria is an indication that HDCA upon becoming bioavailable the systemic circulation is able to induce dissolution of the body fat.

Without wishing to be bound by any particular theory, the elevation of biocompatible emulsifiers in systemic circulation is expected to lead to an increase in the levels of fatty acids in the circulation; which in turn, and as the result of a possible mass balance effect, causes an increased oxidation (burning) of fatty acids in tissues such as muscles and liver. This increased fatty acid oxidation is expected to further lead to activation of hormone sensitive lipase (HSL) which is responsible for release of additional fat from the adipose tissues. Furthermore, the increased in fatty acid oxidation is expected to challenge the processing capacity of the citric acid cycle (TCA cycle) thus causing the excess acetyl-CoA (the end product of fatty acid oxidation) to be shunted towards biosynthesis of ketone bodies (ketogenesis) which are readily excreted by the kidneys as was observed in the individual who took HDCA. Furthermore, without wishing to be bound by any particular theory, the above-described mechanism is expected to function regardless of the subject continuing with his or her normal diet including carbohydrates, although refraining from any or excessive fat or carbohydrate consumption is recommended for subjects who may be treated by the methods or compositions of the present invention.

Accordingly, the present invention provides a novel and useful application of biocompatible emulsifiers for ubiquitous dissolution of fat deposits throughout the body. In some embodiments of the present invention, the systemic use of the biocompatible emulsifiers of the invention (e.g., Hyodeoxycyholic acid) provides ubiquitous distribution of the emulsifiers in the systemic circulation by blood, and therefore, dissolution of fat in the areas not locally accessible by in loco treatment can also take place. Consequently, the compositions and methods of the present invention will not only be advantageous from a cosmetic point of view, but it will provide health benefits associated with a general reduction of fat deposits in the body. Furthermore, with systemic administration of biocompatible emulsifiers, simultaneous dissolution of the fatty aggregates, wherever located in the human body, occurs along with simultaneous impediment to fat deposition and accumulation.

As used in the specification and the appended claims, the singular forms “a,” “an,” and “the” include plural referents unless the context clearly dictates otherwise. Thus, for example, reference to “a pharmaceutically acceptable carrier” includes two or more such carriers as well as a single carrier, and the like.

The term “subject” is meant any subject, generally a mammal (e.g., human, canine, feline, equine, bovine, etc.), in which treatment of overweight or obesity is desired.

The term “obesity” as used herein is a medical condition in which excess body fat has accumulated to the extent that it may have an adverse effect (both physical and mental) on health, leading to reduced life expectancy and/or increased health problems. Body mass index (BMI), a measurement which compares weight and height (calculated from an subject's weight divided by the square of the height), defines people as overweight (pre-obese) when their BMI is between 25 kg/m² and 30 kg/m², and obese when it is greater than 30 kg/m2. Obesity can also be defined as a condition whereby a subject with at least one co-morbidity has a BMI greater than or equal to 27 kg/m². An “obese subject” is an otherwise healthy subject with a Body Mass Index (BMI) greater than or equal to 30 kg/m² or a subject with at least one co-morbidity with a BMI greater than or equal to 27 kg/m². An “overweight” or a “subject at risk of obesity” is an otherwise healthy subject with a BMI of 25 kg/m² to less than 30 kg/m² or a subject with at least one co-morbidity with a BMI of 25 kg/m² to less than 27 kg/m². A skilled artisan would understand that the BMI-based definition of overweight or obesity may be modified to reflect changes in understanding of the condition or practices in the field. Such changes to the BMI-based definition of overweight are contemplated herein. A skilled artisan would also understand that other methods of measurement may be used to define overweight or obesity. Such methods are also contemplated in the present invention. For example, overweight or obesity can be defined in terms of body fat percentage which can be quantified by a variety of means recognized in the art, such as body average density measurement, bioelectric impedance, skinfold method, height and circumference method as well as Body Mass Index.

The increased risks associated with obesity occur at a lower Body Mass Index (BMI) in Asians. In Asian countries, including Japan, “obesity” refers to a condition whereby a subject with at least one obesity-induced or obesity-related co-morbidity, that requires weight reduction or that would be improved by weight reduction, has a BMI greater than or equal to 25 kg/m2. In Asian countries, including Japan, an “obese subject” refers to a subject with at least one obesity-induced or obesity-related co-morbidity that requires weight reduction or that would be improved by weight reduction, with a BMI greater than or equal to 25 kg/m2. In Asia-Pacific, a “subject at risk of obesity” is a subject with a BMI of greater than 23 kg/m2 to less than 25 kg/m2.

As used herein, the term “obesity” is meant to encompass all of the above definitions including the state of being overweight.

Obesity-induced or obesity-related co-morbidities include, but are not limited to, diabetes, non-insulin dependent diabetes mellitus-type 2, diabetes associated with obesity, impaired glucose tolerance, impaired fasting glucose, insulin resistance syndrome, dyslipidemia, hypertension, hypertension associated with obesity, hyperuricacidemia, gout, coronary artery disease, myocardial infarction, angina pectoris, sleep apnea syndrome, Pickwickian syndrome, fatty liver; cerebral infarction, cerebral thrombosis, transient ischemic attack, orthopedic disorders, arthritis deformans, lumbodynia, emmeniopathy, and infertility. In particular, co-morbidities include hypertension, hyperlipidemia, dyslipidemia, glucose intolerance, cardiovascular disease, sleep apnea, diabetes mellitus, and other obesity-related conditions.

As used herein, the phrase “treatment of obesity” or any of its grammatical equivalents refers to the administration of the compounds (i.e., biocompatible emulsifiers) or compositions (including formulations) of the present invention to reduce or maintain the body weight of an obese or overweight subject as defined above. “Treatment of obesity” further refers to the administration of the compounds or compositions of the present invention to an obese or overweight subject, which may result in (a) the reduction of the body weight of the obese or overweight subject relative to that of subject's body weight immediately before the treatment; (b) the reduction of the BMI of the obese or overweight subject relative to that of subject's BMI immediately before the administration of the compounds or compositions of the present invention; (c) the prevention of body weight regain of body weight previously lost as a result of diet, exercise, or pharmacotherapy in the obese or overweight subject; (d) the decrease of the occurrence of and/or the severity of obesity-related diseases or disorders in the obese or overweight person; or (e) the alteration of metabolic rate, such as an increase in metabolic rate, in the obese or overweight subject. The phrase “treatment of obesity” can further encompass prevention of obesity. By “prevention of obesity” it is meant preventing obesity from occurring if the treatment is administered prior to the onset of obesity in a subject at risk of obesity. “Prevention of obesity” can further refer to decreasing the occurrence and/or severity of obesity-related disorders if the treatment is administered prior to the onset of obesity in a subject at risk of obesity. Moreover, if treatment is commenced in already obese subjects, such treatment may prevent the occurrence, progression or severity of obesity-related disorders, such as, but not limited to, arteriosclerosis, Type 2 diabetes, polycystic ovary disease, cardiovascular diseases, osteoarthritis, dermatological disorders, hypertension, insulin resistance, hypercholesterolemia, hypertriglyceridemia, and cholelithiasis.

As used herein, the phrase “biocompatible emulsifier” refer to bile acids, terpenes, saponins or detergents from (naturally available or synthetically prepared) that are suitable and safe for administration to a subject. In particular, the biocompatible emulsifiers of the present invention are those that can enter into and remain in the system blood circulation in the body. Exemplary biocompatible emulsifiers are bile acids such as deoxycholic acid (DCA), hyodeoxycholate (HDCA), ursodeoxycholate (UDCA), D-Limonene, and the like, and their pharmaceutically acceptable salts, conjugates, hydrates, solvates, derivatives, or polymorphs, and mixtures thereof. Examples of biocompatible emulsifiers suitable for use in the present invention such as bile acid emulsifiers, terpene emulsifiers, saponin emulsifiers, or detergent emulsifiers are provided below.

As used herein, the term “bile acid” includes bile acids; pharmaceutically acceptable salts, conjugates, hydrates, solvates, derivatives, or polymorphs of bile acids; and mixtures thereof. As used herein, the term “terpene” includes terpenes; pharmaceutically acceptable salts, conjugates, hydrates, solvates, derivatives, or polymorphs of terpenes; and mixtures thereof. As used herein, the term “saponin” includes saponins; pharmaceutically acceptable salts, conjugates, hydrates, solvates, derivatives, or polymorphs of saponins; and mixtures thereof. As used herein, the term “detergent” includes detergents; pharmaceutically acceptable salts, conjugates, hydrates, solvates, derivatives, or polymorphs of detergents; and mixtures thereof.

As used herein, the phrase “effective amount” or any of its grammatical equivalents refers to the amount of compounds or compositions of the invention which is sufficient for inducing a desired outcome upon administration to an obese or overweight subject. A desired outcome can be reducing the initial weight. Another desired outcome can be reducing the body mass index (BMI) of the subject. Another outcome can be lowering fatty acid and triglyceride levels in the body of the subject. Another outcome can be reducing the effect or severity of one or more of the symptoms associated with obesity in the subject. Another outcome can be altering the metabolic state in the subject.

The phrase “administering to subject” refers to the process of introducing the biocompatible emulsifier of the invention into the subject or patient's body via an art-recognized means of introduction (e.g., orally, transdermally, via injection, etc.). The subject to whom the compounds or compositions of the invention are administered may or may not also be on a weight loss diet regimen. The term “weight loss diet regimen” or related terms, is used broadly here to include any type of nutritional weight loss plan used by a subject. Examples of weight loss diet regimens include, but are not limited to, Atkins diet, Beverly Hills diet, Cabbage Soup diet, DietSmart.com diet, DietWatch.com diet, Fit For Life diet, Grapefruit diet, Herbalife diet, High Protein diet, Jenny Craig diet, Juice Fasts diet, Kashi GoLean diet, Low Fat diet, Mayo Clinic diet, Nutrisystem diet, Perricone diet, Pritkin diet, Ready to Eat diet, Revival Soy diet, Richard Simmons diet, Scarsdale diet, Shakes diet, Slim-Fast diet, Somersizing diet, South Beach diet, Special K diet, Subway diet, Sugar Busters diet, Thin For Life diet, Weight Watchers diet, Zone diet, running, swimming, meditation, yoga, hypnosis, clinical therapy, bicycling, walking, hypnosis, rehabilitory training, a dietary plan provided through a dietician, and surgical procedures.

The terms “composition” or “formulation,” as used interchangeably herein in pharmaceutical composition or formulation, are intended to encompass a product comprising the active ingredient(s) (i.e., one or more biocompatible emulsifiers), and the inert ingredient(s) (pharmaceutically acceptable excipients) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing at least one biocompatible emulsifier compound with additional active ingredient(s) (such as statin) and pharmaceutically acceptable excipients.

As used herein, “systemic circulation” refers to the entirety of components carried along with oxygenated blood by the cardiovascular system as it carries oxygenated blood away from the heart, to the body, and returns deoxygenated blood back to the heart, such as serum, blood plasma, blood cells, red blood cells, white blood cells, antibodies, proteins, nucleic acids, and immune cells.

Results from the 2003-2004 National Health and Nutrition Examination Survey estimates that about 65% of US adults are either overweight or obese. Another recent estimate indicates that about 18.9% of the total US population and 31% of US adults are obese. The prevalence of overweight among children in the US has been more than doubled between the 1960s and 1988-1994 and is expected to further increase beyond 2010. In 2007, about 1.7 billion people worldwide were overweight or obese. An overweight person has a body mass index (BMI) between 25.0-29.9 and an obese subject has a BMI greater than or equal to 30.0. BMI is expressed as weight in kilograms (or pounds) divided by their height in meters (or inches) squared (Sonnenberg et al., Drug treatments for obesity: where are we heading and how do we get there?, Br. J. Diabetes Vasc. Dis. 7, 111-118 (2007); Ogden et al., Prevalence and trends in overweight among US children and adolescents, 1999-2000, JAMA 288(14), 1728-1732 (2002)).

Overeating and reduced physical activity are considered to be the two major reasons of obesity, although several additional factors may also contribute. Unfortunately, some of the frequently used anti-diabetic agents also cause weight gain for which the best example is insulin. Other anti-diabetic agents causing weight gain include the thiazolidinedione class of compounds (such as Rosiglitazone and Pioglitazone), the sulfonylurea class of drugs (Tolbutamide, Glyburide), and Repaglinide (a meglitinide class drug). Thus, in a sense, while these drugs are useful by promoting removal of excess glucose from the blood, they also add to the problem by increasing adiposity. Other drugs that may enhance weight gain include atypical antipsychotics (olanzapine, clozapine), antidepressants, mood stabilizers, anticonvulsants, steroid hormones, beta-blockers, oral contraceptives, antihistamines, HIV antiretroviral drugs and protease inhibitors (Keith, S. W. et al., Putative contributors to the secular increase in obesity: exploring the roads less traveled, International. J. Obesity. 30, 1585-1594 (2006)).

Obesity is a risk factor for many diseases and unhealthy conditions including high blood pressure, insulin resistance or impaired glucose tolerance, hyperinsulinemia, metabolic syndrome, type 2 diabetes that with time may transition into type 1 diabetes, stroke, heart attack, heart failure, atherosclerosis, inflammation, coagulation, fibrinolysis, certain types of cancer, gallstones, gout and gouty arthritis, osteoarthritis, sleep apnea, pickwickian syndrome, periodontal disease, abdominal hernias, varicose veins, renal failure, dementia, and liver malfunction. Obesity is also an independent predictor of mortality following severe blunt trauma (Neville et al., Obesity is an independent risk factor of mortality in severely injured blunt trauma patient, Arch. Surg. 139, 983-987 (2004)). One of the most devastating consequences of obesity is diabetes; about 60% of all type 2 diabetes cases is due to obesity (Runge, C. F., Economic consequences of the obese, Diabetes 56, 2668-2672 (2007)). Because of the risks obesity entails, someone who is 40% overweight is twice as likely to die prematurely compared to an average-weight person.

The private and social costs of obesity are large. According to a recent estimate, health care costs for overweight and obese individuals are 37% higher than for people of normal weight, adding an extra $ 732 to the health care bill of each and every American. In the US, obesity and related conditions are estimated to result in $62.7 billion in doctor's visits and $ 39.3 billion in lost workdays each year (Runge, C. F., Economic consequences of the obese, Diabetes 56, 2668-2672 (2007)).

One of the current obesity treatment options includes reduced caloric intake and increased physical activity. These approaches however fail in 90% of cases. Studies show that diets and exercise programs initially result on average in about 10% weight loss, but obese subjects tend to regain two-thirds of weight within one year and most of the weight within five years. Another method to help lose weight is bariatric surgery that could lead to 21-38% loss of baseline weight. However, this procedure is accompanied with surgical-related risks and requires a life time adherence to extensive dietary, exercise, and medical guidelines.

Phentermine is an appetite suppressant that is recommended for short-term use because of its possible side effects. In addition, a limited number of anti-obesity drugs are available for longer-term use. These include Orlistat that inhibits intestinal lipase enzymes, and Sibutramine that blocks re-uptake of monoamine neurotransmitters in the brain. Rimonabant (Acomplia) has been widely used in Europe during the last few years but it has not received the U.S. Food and Drug Administration's approval because of psychiatric effects. Rimonabant blocks binding of endogenous cannabinoid to neuronal CB1 receptors. Each drug has side effects such as diarrhea, flatulence, bloating, abdominal pain, and sispepsia (Orlistat); dry mouth, constipation and insomnia (Sibutramine); nausea and mood disorders (Rimonabant).

Accordingly, there remains a need for non-invasive, systemically effective compositions and methods effective to result in the treatment of obesity.

It is thus an object of the present invention to provide pharmacological compounds or compositions capable of dissolving/emulsifying ultimately causing lipolysis of the lipid deposits ubiquitously and to prevent formation/accumulation of new fat. It is also an object of the present invention to provide a biocompatible pharmacological compound and compositions that, by removing the fat deposits and by preventing its deposition and accumulation, contribute to the minimization of the complications associated with obesity. As such, the invention provides a biocompatible emulsifier such as a surfactant or detergent alone or in combination with other biocompatible emulsifiers for administration to a subject via routes which make such biocompatible emulsifier(s) (alone or in combination) available in the systemic blood circulation of the subject for the purpose of ubiquitously dissolving the lipid deposits and preventing new fat deposit formation. The compounds of the present invention (i.e., biocompatible emulsifiers) can be used alone or in combination with other emulsifiers of different nature or origin or in combination with lipolytic biocompatible substances such as the lipase group of enzymes, and with compounds enhancing transport and/or catabolism of the dissolved fat.

The targets of pharmacological action of the compounds and compositions of the present invention are contemplated to be the pre-existing fatty deposits of the fat tissues or fats in blood circulation in any shape or form, such as Low Density Lipoproteins, chylomicrons, micelles or fat droplets or fat globules, which although they represent a form of dissolved fat in the circulating blood, they may require finer emulsification into a sort of Very Low Density Lipoproteines in order to be utilized by oxidative catabolic processes, such finer emulsification being provided by the compounds and compositions of the present invention.

Exemplary biocompatible emulsifiers that can be used in the compositions and methods of the present invention include, for example, hyodexoycholic acid (HDCA), dexycholic acid (DCA), ursodeoxycholic acid (UDCA), D-limonene, or detergents classified according to structure such as (i) alkyl glycosides: n-nonyl-β-D-glucopyranoside, n-octyl-β-D-glucopyranoside, n-heptyl-β-D-glucopyranoside, n-hexyl-β-D-glucopyranoside, dodecyl-β-D-maltoside, decyl-β-D-maltoside, octyl-β-Dthioglucopyranoside, and others; (ii) glucamides such as MEGA-10, MEGA-9, MEGA-8, Deoxy Big CHAP, Big CHAP, and others; (iii) polyoxyethylenes, monodisperse and polydisperse such as reduced TRITON® X-100; reduced TRITON® X-114; TRITON® X-100; NP-40; TRITON® X-114; GENAPOL® X-080; GENAPOL® X-100; C12E8; C12E9; THESIT®; LUBROL® PX; GENAPOL® C-100; BRIJ® 35; PLURONIC® F-127®; (laurate); TWEEN® 20 (oleate); TWEEN® 80; and others; (iv) ionic detergents such as BATC; Cetyltrimethylammonium Bromide (CTAB), Molecular Biology Grade; Chenodeoxycholic Acid, Free Acid; Chenodeoxycholic Acid, Sodium Salt; Cholic Acid, Sodium Salt; Cholic Acid, Sodium Salt, ULTROL® Grade; Deoxycholic Acid, Sodium Salt; Deoxycholic Acid, Sodium Salt, ULTROL® Grade; 7a,12a-Dihydroxy-5β-cholanic Acid; Glycholic Acid, Sodium Salt; Glycodeoxycholic Acid, Sodium Salt; Lauroylsarcosine, Sodium Salt; Sodium n-Dodecyl Sulfate (SDS); Sodium n-Dodecyl Sulfate (SDS), High Purity; Sodium n-Dodecyl Sulfate (SDS), Molecular Biology Grade; Sodium n-Dodecyl Sulfate (SDS), 30% Solution; Taurochenodeoxycholic Acid, Sodium Salt; Taurocholic Acid, Sodium Salt; Taurocholic Acid, Sodium Salt, ULTROL® Grade; Taurodehydrocholic Acid, Sodium Salt; Taurodeoxycholic Acid, Sodium Salt; Taurolithocholic Acid, Sodium Salt; Tauroursodeoxycholic Acid, Sodium Salt; TOPPS; (v) non-ionic Detergents such as APO-10; APO-12; Big CHAP; Big CHAP, Deoxy; BRIJ® 35; PROTEIN GRADE® Detergent, 30% Solution; BRIJ® 35, PROTEIN GRADE® Detergent, 10% Solution, Sterile-Filtered; C12E6; C12E8; C12E9; Cyclohexyl-n-ethyl-β-D-maltoside; LTROL® Grade; Cyclohexyl-n-hexyl-β-D-maltoside, ULTROL® Grade; Cyclohexyl-n-methyl-β-D-maltoside, ULTROL® Grade; n-Decanoylsucrose; n-Decyl-β-D-maltopyranoside; ULTROL® Grade 252718; n-Decyl-β-D-thiomaltoside, ULTROL® Grade; Digitonin, High Purity; Digitonin, Alcohol-Soluble, High Purity; n-Dodecanoylsucrose 324374; n-Dodecyl-β-D-glucopyranoside 324355; ELUGENT™ Detergent, 50% Solution; GENAPOL® C-100, PROTEIN GRADE® Detergent, 10% Solution; GENAPOL® X-80, PROTEIN GRADE® Detergent, 10% Solution; GENAPOL® X-100, PROTEIN GRADE® Detergent, 10% Solution; n-Heptyl-β-D-glucopyranoside; n-Heptyl-β-D-thio glucopyranoside, ULTROL® Grade, 10% Solution; n-Hexyl-β-D-glucopyranoside; MEGA-8, ULTROL® Grade; MEGA-9, ULTROL® Grade; MEGA-10, ULTROL® Grade; n-Nonyl-β-D-glucopyranoside; NP-40, PROTEIN GRADE® Detergent, 10% Solution; n-Octanoyl-β-D-glucosylamine (NOGA); n-Octanoylsucrose; n-Octyl-β-D-glucopyranoside; n-Octyl-β-D-glucopyranoside; ULTROL® Grade; n-Octyl-β-D-maltopyranoside; n-Octyl-β-D-thioglycopyranoside; PLURONIC® F-127, PROTEIN GRADE® Detergent, 10% Solution; TRITON® X-100; PROTEIN GRADE® Detergent, 10% Solution; TRITON® X-100, Molecular Biology Grade; TRITON® X-100, Hydrogenated; TRITON® X-100, Hydrogenated, PROTEIN GRADE® Detergent, 10% Solution; TRITON® X-114, PROTEIN GRADE® Detergent, 10% Solution; TWEEN® 20; TWEEN® 20, Molecular Biology Grade; TWEEN® 20, PROTEIN GRADE® Detergent, 10% Solution; TWEEN® 80, PROTEIN GRADE® Detergent, 10% Solution; or n-Undecyl-β-D-maltoside, ULTROL® Grade and others; (vi) zwitterionic detergents such as ASB-14; ASB-16; CHAPS; CHAPSO; DDMAB; DDMAU; EMPIGEN; BBC) Detergent, 30% Solution; Lauryldimethylamine Oxide (LDAO), 30% Solution; ZWITTERGENT® 3-08 Detergent; ZWITTERGENT® 3-10 Detergent; ZWITTERGENT® 3-12 Detergent; ZWITTERGENT® 3-14 Detergent; ZWITTERGENT® 3-16 Detergent; and others.

Other exemplary biocompatible emulsifiers are saponins from plat sources such as Soapberry and many other members of the family Sapindaceae, including buckeyes Soapwort, conkers/horse chestnuts, Digitalis as digitonin, Grape skin, Olives, Panax as Ginsenoside including Panax Notoginseng rich in saponins content, Gymnostemma Pentaphyllum, Quillaja Saponaria which is member of the Rosaceae family, Soybeans, Yucca, Aloe, Quinoa, Bacopa monnieri, Chlorophytum species, Chlorogalum species soap plants, Tuberous cucurbit species, Medicago sativa, chickpeas Cicer arietinum, seed and foliage, common beans, several rangeland weeds in the US including corn cockle Agrostemma Githago, broomweed (Gutierrezia Sarothrae), Alfombrilla (Drymaria arenaroides), Christmas Rose, Helleborus niger, Asparagus fern, Asparagus officinalis, Daisies Bellis perennis, Dioscorea spp, Honeylocust, Fenugreek, Platycodon species, Glycyrrhiza glabra and many others including the saponins derived from Vitis Vinifera (Grapes skin). Cuticular wax has been shown to contain saponins. The saponins discovered in the wines contain ursolic acid, oleanolic acid, ursolic aldehyde, oleanolic aldehyde, hydroxyhopanone, damarenolic acid, mastidienonic acid isomasticadienonic acid. The Vitis Vinifera saponins can be used alone or in association with phenolic compounds such as Resveratrol.

Some embodiments of the present invention provide pharmaceutical compositions comprising one or more biocompatible emulsifiers, in an effective amount, administered to treat obesity in a subject. Such emulsifiers include bile acids, terpenes, saponins, detergents, or combinations thereof.

Bile acids are cholesterol-derived organic acids that have detergent properties. Bile acids play important roles physiologically in the digestion, absorption, transport, and secretion of lipids. Bile acids are involved in intestinal lipid digestion, by promoting fine emulsification of lipids, which enhances the exposure of lipids to lipid-digesting enzymes, such as pancreatic lipases. In addition to being direct emulsifiers of lipids, bile acids can also function to directly activate (e.g., allosteric effectors) lipases, such as cholesteryl ester hydrolase, that can be found in fat tissues. Therefore, in some embodiments, bile acids can emulsify short chain fatty acids released from fat tissues by lipase enzymatic activity and induce weight loss, which will result in the treatment of obesity.

Bile acids can be classified as primary or secondary bile acids, depending on whether they are synthesized de novo (primary) or are derived by subsequent chemical modification (secondary). Primary bile acids are produced by the liver and include cholic acid (3α, 7α, 12α,-trihydroxy-5β-cholanic acid) and chenodeoxycholic acid (3α, 7α,-dihydroxy-β-cholanic acid). Dehydroxylation of the primary bile acids, for example by intestinal bacteria, produces the more hydrophobic secondary bile acids, for example deoxycholic acid (3α, 12α,-dihydroxy-5β-cholanic acid), and lithocholic acid (3α-hydroxy-5β-cholanic acid). Together, the primary and secondary bile acids make up about 99% of the total bile acid pool in humans.

The embodiments of the present disclosure teach formulations and methods that lead to a sustained increase in the level of biocompatible empulsifiers such as bile acid and/or bile salt emulsifiers in the systemic circulation effective to treat obesity. Experimental examples described below demonstrate that bile acid emulsifiers are effective to induce ketonuria with is indicates increased fatty acid metabolism in the body as a sign of metabolic alteration and weight loss necessary or sufficient for treating obesity.

There are instances where the concentration of bile acids have been increased systemically. For example, it has been previously shown that feeding hyodeoxycholic acid (HDCA) to C57BL/6 LDL r-KO knockout mice (genetically predisposed to develop atherosclerosis) results in a reduced rate of formation of atherosclerotic plaque relative to mice not provided HDCA (Sehayek et al., J. Lip. Res. 42: 1250-1256, 2001). Plasma levels of wild-type mice, provided the same amount of dietary HDCA, ranged up to about 50 μM. However, there is no evidence that these levels were effective to result in induction of weight loss or prevention of weight gain, as provided by certain embodiments described herein.

As described in Example 2, oral administration of 250 mg of HDCA three time a day resulted in a mild ketotic state indicative of an induced fat metabolism and degradation in and removal from the body. Considering that about 70% of HDCA escapes the entero-hepatic pathway and enters into the systemic blood circulation, the 750 mg/day dose of HDCA averages to concentration of about 270 μM in the blood of an average adult person weighing 70 kg, as compared to normal levels of bile salts in the blood which are less than 10 μM. Some embodiments described herein are effective to mimic the high levels of bile salts observed as safe in Example 2, and in so doing are effective to result in the treatment of obesity.

In some embodiments, administration schedules of a pharmaceutical composition or formulation of the present invention comprising a biocompatible emulsifier such as bile acids, terpenes, saponins, and/or detergents effective to treat obesity involve administering the composition or formulation once per day, twice per day, three times per day, four times per day, five times per day, six times per day, seven times per day, eight times per day, nine times per day, 10 times per day, 11 times per day, 12 times per day, 13 times per day, 14 times per day, 15 times per day, 16 times per day, 17 times per day, 18 times per day, 19 times per day, 20 times per day, 21 times per day, 22 times per day, 23 times per day, 24 times per day, and continuously. In some embodiments, daily or continuous administration of a pharmaceutical formulation of the present invention may comprise a period of at least one day, two days, three days, four days, five days, six days, seven days, two weeks, three weeks, one month, two months, three months, four months, five months, six months, seven months, eight months, nine months, 10 months, 11 months, one year, two years, three years, four years, and five years. In some embodiments, daily or continuous administration of the pharmaceutical formulation may be intermittent within an administration period, for instance, every other day, every third day, every fourth day, every fifth day, every sixth day, once a week, once every two weeks, once every three weeks, once a month, once every two months, once every three months, once every four months, once every five months, once every six months, once every seven months, once every eight months, once every nine months, once every 10 months, once every 11 months, and once a year.

In some embodiments, an effective dose of a pharmaceutical formulation results in elevated levels of biocompatible emulsifiers such as bile acids, terpenes, saponins, and/or detergents in the systemic circulation sustained for a period of, for instance, at least about one hour, at least about two hours, at least about three hours, at least about four hours, at least about five hours, at least at least about six hours, at least about seven hours, at least about eight hours, at least about nine hours, at least about 10 hours, at least about 11 hours, at least about 12 hours, at least about 13 hours, at least about 14 hours, at least about 15 hours, at least about 16 hours, at least about 17 hours, at least about 18 hours, at least about 19 hours, at least about 20 hours, at least about 21 hours, at least about 22 hours, at least about 23 hours, and at least about 24 hours.

In some embodiments, an effective dose of a pharmaceutical formulation comprising a biocompatible emulsifier or a combination of emulsifiers results in reduction of the initial body weight by at least 5% in a certain period of time. In some other embodiments, the an effective dose of a pharmaceutical formulation comprising a biocompatible emulsifier or a combination of emulsifiers results in reduction of the initial body weight by at least 7%, or at least 9%, or at least 11%, or at least 13%, or at least 15%, or at least 17%, or at least 19%, or at least 21%, or at least 25%, or at least 30%, or at least 35%, or at least 40%. In some other embodiments, the an effective dose of a pharmaceutical formulation comprising a biocompatible emulsifier or a combination of emulsifiers results in reduction of the initial body weight by the percentages listed above in two weeks, three weeks, four weeks, six weeks, eight weeks, three months, four months, five months or more.

In some embodiments, an effective dose of a pharmaceutical formulation comprising a biocompatible emulsifier or a combination of emulsifiers results in weight loss that is greater than the placebo effect, and the mean formulation-associated weight loss exceeds the mean placebo weight loss by at least 5% as recommended by the FDA Draft Guidelines for the Clinical Evaluation of Weight Control Drugs (1994) available at www.fda.gov/OHRMS/DOCKET S/98fr/03d-0570-gd10001.pdf (last viewed October 2010).

In some embodiments, an effective dose of a pharmaceutical formulation comprising a biocompatible emulsifier or a combination of emulsifiers results in weight loss wherein the proportion of subjects who reach and maintain a loss of at least 5% of their initial body weight is significantly greater in subjects on pharmaceutical formulations of the present invention than in those on placebo as recommended by the FDA Draft Guidelines for the Clinical Evaluation of Weight Control Drugs (1994) available at www.fda.gov/OHRMS/DOCKETS/98fr/03d-0570-gd10001.pdf (last viewed October 2010).

In some embodiments, sustained levels of an effective dose of a pharmaceutical formulation comprising a biocompatible emulsifier or a combination of emulsifiers of the present invention is effective to reduce excess body fat (accumulated in adipose tissue) by, for instance, about 1% to about 5%, about 5% to about 10%, about 10% to about 20%, about 20% to about 30%, about 30% to about 40%, about 40% to about 50%, about 50% to about 60%, about 60% to about 70%, about 70% to about 80%, about 80% to about 90%, and about 90% to about 100%.

In some embodiments of the present invention, a biocompatible emulsifier or a combination of emulsifiers of the present invention is in an amount effective to result in a serum concentration of greater than 50 μM, or result in a serum concentration of about 60 μM, about 60 μM to about 70 μM, about 70 μM to about 80 μM, about 80 μM to about 90 μM, about 90 μM to about 100 μM, about 50 μM to about 600 μM, about 50 μM to about 100 μM, about 100 μM to about 300 μM, about 100 μM to about 550 μM, about 150 μM to about 1M, about 150 μM to about 1 mM, about 150 μM to about 500 μM, about 200 μM to about 450 μM, about 250 μM to about 400 μM, about 300 μM to about 350 μM, about 500 μM to about 600 μM, about 600 μM to about 700 μM, about 700 μM to about 800 μM, about 800 μM to about 900 μM, about 900 μM to about 1 mM, about 1 mM to about 100 mM, about 100 mM to about 200 mM, about 200 mM to about 300 mM, about 300 mM to about 400 mM, about 400 mM to about 500 mM, about 500 mM to about 600 mM, about 600 mM to about 700 mM, about 700 mM to about 800 mM, about 800 mM to about 900 mM, and about 900 mM to about 1 M.

In some embodiments of the present invention, the subject being treated by the methods or compositions of the present invention is consuming a diet restricted in carbohydrates to less than 100 grams per day, or 80 grams per day, or 60 grams per day, or 40 grams per day, or 20 grams per day, or 5 grams per day or 1 gram per day. Alternatively or in addition, the subject being treated is on a weight loss diet regimen which may include any type of nutritional weight loss plan used by a subject. Examples of weight loss diet regimens include, but are not limited to, Atkins diet, Beverly Hills diet, Cabbage Soup diet, DietSmart.com diet, DietWatch.com diet, Fit For Life diet, Grapefruit diet, Herbalife diet, High Protein diet, Jenny Craig diet, Juice Fasts diet, Kashi GoLean diet, Low Fat diet, Mayo Clinic diet, Nutrisystem diet, Perricone diet, Pritkin diet, Ready to Eat diet, Revival Soy diet, Richard Simmons diet, Scarsdale diet, Shakes diet, Slim-Fast diet, Somersizing diet, South Beach diet, Special K diet, Subway diet, Sugar Busters diet, Thin For Life diet, Weight Watchers diet, Zone diet, running, swimming, meditation, yoga, hypnosis, clinical therapy, bicycling, walking, hypnosis, rehabilitory training, a dietary plan provided through a dietician, and surgical procedures.

As described above, the biocompatible emulsifiers of the present invention include bile acids, terpenes, saponins or biocompatible detergents. Specific examples of each of these emulsifiers are provided below:

### Examples of Bile Acid/Bile Salt Emulsifiers

Examples of bile acids useful in certain embodiments described herein can include, without limitation any naturally occurring or synthetically produced bile acid, salt, or conjugate thereof, having the ability to induce fatty acid oxidation and weight loss. This can include cholic acid, chenodeoxycholic acid, deoxycholic acid (DCA), lithocholic acid, ursodeoxycholic acid (UDCA), hyodeoxycholic acid (HDCA), and any conjugate or pharmaceutically acceptable salt thereof.

In addition, bile acids useful in certain embodiments of formulations for use as described herein can include, without limitation: 1,3,12-trihydroxycholanoic acid; 1,3,7,12-tetrahydroxycholanoic acid; 3beta-hydroxy-delta 5-cholenic acid; 3 beta-hydroxychol-3-en-24-oic acid; 3′-isøthiocyanatobenzamidecholic acid; 3,12-dihydroxy-5-cholenoic acid; 3,4,7-trihydroxycholanoic acid; 3,6,12-trihydroxycholanoic acid; 3,7,12,23-tetrahydroxycholan-24-oic acid; 3,7,12-trihydroxy-7-methylcholanoic acid; 3,7,12-trihydroxycoprostanic acid; 3,7,23-trihydroxycholan-24-oic acid; 3,7-dihydroxy-22,23-methylene-cholan-24-oic acid (2-sulfoethyl)amide; 3-((3-cholamidopropyl)dimethylammonium)-1-propanesulfonate; 3-((3-deoxycholamidopropyl)dimethylammonio)-1-propane; 3-benzoylcholic acid; 3-hydroxy-5-cholen-24-oic acid 3-sulfate ester; 3-hydroxy-7-(hydroxyimino)cholanic acid; 3-Iodocholic acid; 7,12-dihydroxy-3-(2-(glucopyranosyl)acetyl)cholan-24-oic acid; 7,12-dihydroxy-3-oxocholanic acid; allocholic acid; chapso; chol-3-en-24-oic acid; cholanic acid; sodium cholate; methyl cholate; benzyldimethylhexadecylammonium cholate; methyl 1,3-dihydroxycholan-24-o ate; and trioctylmethylammonium cholate); cholic acid glucuronide; cholyl-coenzyme A; cholyl-lysylfluorescein; cholyldiglycylhistamine; cholylhistamine; cholylhydroxamic acid; cholylsarcosine; cholyltetraglycylhistamine; ciliatocholic acid; dehydrocholic acid (which includes FZ 560; Gallo-Merz; Gillazym; Hepavis; Mexase; progresin Retard; and spasmocanulase); 23-nordeoxycholic acid; 3,7-dioxocholanoic acid; 3-hydroxy-polydeoxycholic acid; 3-sulfodeoxycholic acid; 6-hydroxycholanoic acid; 6-methylmurideoxycholic acid; 7-ketodeoxycholic acid; 7-methyldeoxycholic acid; chenodeoxycholic acid; dehydrodeoxycholic acid; deoxycholyltyrosine; desoxybilianic acid; glycodeoxycholic acid; hyodeoxycholate-6-O— glucuronide; hyodeoxycholic acid; taurodeoxycholic Acid; and ursodeoxycholic acid; glycocholic acid; 3-hydroxy-5-cholenoylglycine; cholylglycylhistamine; cholylglycyltyrosine; glycodeoxycholic Acid; sulfolithocholylglycine; hemulcholic acid; 12-ketolithocholic acid; 24-norlithocholic acid; 3-dehydrolithocholylglycine; 3-hydroxy-6-cholen-24-oic acid; 3-hydroxy-7,12-diketocholanoic acid; 3-hydroxy-7-methylcholanoic acid; 3-ketolithocholic acid; 3-oxochol-4-en-24-oic acid; 3-oxocholan-24-oic acid; 4-azidophenacyl lithocholate; 7-ketolithocholic acid; BRL 39924A; glycolithocholic acid; lithocholate 3-O— glucuronide; lithocholyl-N-hydroxysuccinimide; methyl lithocholate; N-carbobenzoxy-N-lithocholyl-epsilon-lysine; N-epsilon-lithochoiyllysine; sulfolithocholic acid; and taurolithocholic acid; muricholic acid; N-(1,3,7,12-tetrahydroxycholan-24-oyl)-2-aminopropionic acid; N-(2-aminoethyl)-3,7,12-trihydroxycholan-24-amide; N-carboxymethyl)-N-(2-(bis(carboxymethypamino)ethyl)-3-(4-(N′-(2-((3,7,12-trihydroxycholan-24-oyl)araino)ethyl)(thioureido)phenyl)alanine; N-cholyl-2-fluoro-beta-alanine; norcholic acid; norursocholic acid; taurocholic acid; (N-(7-(nitrobenz-2-oxa-1,3-diazol-4-yl))-7-amino-3alpha, 12alpha-dihydroxycholan-24-oyl)-2-aminoethanesulfonate; 23-seleno-25-homotaurocholic acid; 3,12-dihydroxy-7-oxocholanoyltaurine; 3-hydroxy-7-oxocholanoyltaurine; azidobenzamidotaurocholate; hexadecyltributylammonium taurocholate; tauro 1-hydroxycholic acid; tauro-3,7-dihydroxy-12-ketocholanoic acid; taurodehydrocholate; taurodeoxycholic acid; tauroglycocholic acid; taurolithocholic acid; tauromurichoUc acid; tauronorcholic acid); tetrahydroxy-5-cholan-24-oic acid; ursocholic acid; vulpecholic acid; bile acid sulfates; glycodeoxycholic acid; glycochenodeoxycholic acid; 7-oxoglycochenodeoxycholic acid; glycochenodeoxycholate-3-sulfate; glycohyodeoxycholic acid; tauro-7,12-dihydroxycholanic acid; tauro chenodeoxycholic acid; tauro chenodeoxycholate-3-sulfate; tauro chenodeoxycholate-7-sulfate; tauroursodeoxycholic acid; taurohyodeoxycholic acid; the includes: 23-methylursodeoxycholic acid; 24-norursodeoxycholic acid; 3,6-dihydroxy-6-methylcholanoic acid; 3,7-dihydroxy-20,22-methylenecholan-23-oic acid; 3,7-dihydroxy-22,23-methylenecholan-24-oic acid; 3,7-dihydroxy-7-ethylcholanoic acid; 3,7-dihydroxy-7-methylcholanoic acid; 3,7-dihydroxy-7-n-propylcholanoic acid; Bamet-UD2; diammhiebis(ursodeoxycholate(O,O′))ρIatinum(II); glycoursodeoxycholic acid; homoursodeoxycholic acid; HS 1030; HS 1183; isoursodeoxycholic acid; PABA-ursodeoxycholic acid; sarcosylsarcoursodeoxycholic acid; sarcoursodeoxycholic acid; ursodeoxycholate-3-sulfate; ursodeoxycholic acid 7-oleyl ester; ursodeoxycholic acid N-acetylglucosaminide; ursodeoxycholic acid-3-O-glucuronide; ursodeoxycholyl N-carboxymethylglycine; ursodeoxycholylcysteic acid; ursometh; 24-norchenodeoxycholic acid; 3,7-dihydroxy-12-oxocholanoic acid; 3,7-dihydroxy-24-norcholane-23-sulfonate; 3,7-dihydroxy-25-homocholane-25-sulfonate; 3,7-dihydroxychol-5-enoic acid; 3,7-dihydroxycholane-24-sulfonate; 3-glucosido-chenodeoxycholic acid; 3-oxo-7-hydroxychol-4-enoic acid; 6-ethylchenodeoxycholic acid; chenodeoxycholate sulfate conjugate; chenodeoxycholyltyrosine; glycochenodeoxycholic acid which includes: 7-oxoglycochenodeoxycholic acid and glycochenodeoxycholate-3-sulfate; homo chenodeoxycholic acid; HS 1200; methyl 3,7-dihydroxychol-4-en-24-oate; methyl 3,7-dihydroxycholanate; N-(2-aminoethyl)-3,7-dihydroxycholan-24-amide; N-chenodeoxycholyl-2-fluoro-beta-alanine; sarcochenodeoxycholic acid; tauro chenodeoxycholic acid; taurochenodeoxycholate-3-sulfate; taurochenodeoxycholate-7-sulfate; or tauroursodeoxycholic acid and the like.

In some embodiments, fatty acids conjugated to bile acids/salts useful in certain embodiments of formulations for use as described herein can include, without limitation butyric acid, caproic acid, caprylic acid, lauric acid, myristic acid, palmitic acid, stearic acid, arachidic acid, behenic acid, oleic acid, linoleic acid, alpha-linolenic acid, arachidonic acid, eicosapentaenoic acid, docosahexaenoic acid, and euric acid.

### Examples of Serum and Systemic Circulation Concentrations of Bile Acid Emulsifiers

Serum and systemic circulation concentrations of a bile acid effective to treat obesity may vary depending on a number of factors. Influential variables can include, for example, various chemical properties of one bile acid, as compared to another. For example different bile acids can differ in pKₐ, solubility, molecular weight, etc., and these properties of a particular bile acid may affect how a patient metabolizes the bile acid, how much of the bile acid enters and remains in the systemic circulation of a subject, and how effectively the bile acid treats obesity.

Accordingly, in some embodiments of the present invention, a serum or a systemic circulation concentration of a bile acid (e.g., HDCA) effective to treat obesity may be greater than 50 μM, or may be in a range of from, for instance, about 60 μM to about 70 μM, about 70 μM to about 80 μM, about 80 μM to about 90 μM, about 90 μM to about 100 μM, about 50 μM to about 600 μM, about 50 μM to about 100 μM, about 100 μM to about 300 μM, about 100 μM to about 550 μM, about 150 μM to about 1 M, about 150 μM to about 1 mM, about 150 μM to about 500 μM, about 200 μM to about 450 μM, about 250 μM to about 400 μM, about 300 μM to about 350 μM, about 500 μM to about 600 μM, about 600 μM to about 700 μM, about 700 μM to about 800 μM, about 800 μM to about 900 μM, about 900 μM to about 1 mM, about 1 mM to about 100 mM, about 100 mM to about 200 mM, about 200 mM to about 300 mM, about 300 mM to about 400 mM, about 400 mM to about 500 mM, about 500 mM to about 600 mM, about 600 mM to about 700 mM, about 700 mM to about 800 mM, about 800 mM to about 900 mM, and about 900 mM to about 1 M.

### Examples of Bile Acid Doses

In some embodiments, a bile acid dose effective to treat obesity may be, in weight of administered bile acid per kilogram of subject body weight per day (mg/kg/day), in a range of from, for instance, about 1 mg/kg/day to about 10 mg/kg/day, about 10 mg/kg/day to about 20 mg/kg/day, about 20 mg/kg/day to about 30 mg/kg/day, about 30 mg/kg/day to about 40 mg/kg/day, about 40 mg/kg/day to about 50 mg/kg/day, about 50 mg/kg/day to about 60 mg/kg/day, about 60 mg/kg/day to about 100 mg/kg/day, about 100 mg/kg/day to about 125 mg/kg/day, about 125 mg/kg/day to about 150 mg/kg/day, about 150 mg/kg/day to about 175 mg/kg/day, about 175 mg/kg/day to about 200 mg/kg/day, about 200 mg/kg/day to about 225 mg/kg/day, about 225 mg/kg/day to about 250 mg/kg/day, about 250 mg/kg/day to about 275 mg/kg/day, about 275 mg/kg/day to about 300 mg/kg/day, about 300 mg/kg/day to about 325 mg/kg/day, about 325 mg/kg/day to about 350 mg/kg/day, about 350 mg/kg/day to about 375 mg/kg/day, about 375 mg/kg/day to about 400 mg/kg/day, about 400 mg/kg/day to about 425 mg/kg/day, about 425 mg/kg/day to about 450 mg/kg/day, about 450 mg/kg/day to about 475 mg/kg/day, about 475 mg/kg/day to about 500 mg/kg/day, about 500 mg/kg/day to about 550 mg/kg/day, about 550 mg/kg/day to about 600 mg/kg/day, about 600 mg/kg/day to about 650 mg/kg/day, about 650 mg/kg/day to about 700 mg/kg/day, about 700 mg/kg/day to about 750 mg/kg/day, about 750 mg/kg/day to about 800 mg/kg/day, about 800 mg/kg/day to about 850 mg/kg/day, about 850 mg/kg/day to about 900 mg/kg/day, about 900 mg/kg/day to about 950 mg/kg/day, about 950 mg/kg/day to about 1 g/kg/day, about 1 g/kg/day to about 1.25 g/kg/day, about 1.25 g/kg/day to about 1.5 g/kg/day, about 1.5 g/kg/day to about 1.75 g/kg/day, about 1.75 g/kg/day to about 2 g/kg/day, about 2 g/kg/day to about 2.25 g/kg/day, about 2.25 g/kg/day to about 2.5 g/kg/day, about 2.5 g/kg/day to about 2.75 g/kg/day, about 2.750 g/kg/day to about 3 g/kg/day, about 3 g/kg/day to about 4 g/kg/day, about 4 g/kg/day to about 5 g/kg/day, about 5 g/kg/day to about 6 g/kg/day, about 6 g/kg/day to about 7 g/kg/day, about 7 g/kg/day to about 8 g/kg/day, about 8 g/kg/day to about 9 g/kg/day, about 9 g/kg/day to about 10 g/kg/day, about and 10 g/kg/day to about 20 g/kg/day.

### Examples of Terpene Emulsifiers

Examples of terpene emulsifiers useful in certain embodiments described herein can include any naturally occurring or synthetically produced terpene, and/or terpene metabolite. Terpenes can be synthesized, and can also be found in nature, for instance, in plant essential oils. Terpenes comprise an isoprene building block, CH₂═C(CH₃)—CH═CH₂, and can comprise a basic molecular formula of (C₅H₈)ₙ and derivatives thereof, in which n is the number of linked isoprene units. The isoprene units of terpenes may be linked together “head to tail” to form linear chains or they may be arranged to form rings. As used herein, terpenes may comprise isoprene units modified with oxygen-containing compounds such as alcohols, aldehydes or ketones.

Hemiterpenes comprise a single isoprene unit, and an example of a hemiterpene is isoprene. Monoterpenes comprise two isoprene units, and examples of monoterpenes include menthol, gerinol, limonene, D-limonene, L-limonene, and terpinol. Metabolites of monopterpenes include S-perillic acid. Sesqueterpenes comprise three isoprene units, and examples of sesquiterpenes include farnesol. Diterpenes comprise four isoprene units, and are derived from geranylgeranyl phosphate. Examples of diterpenes include cafestol, kahweol, cembrene, and taxadiene, (precursor of Taxol). Diterpenes also form the basis for compounds such as retinol, retinal, and phytol. The herb sidiritis contains diterpenes. Sesterterpenes comprise five isoprene units. Triterpenes comprise six isoprene units, tetraterpenes contain eight isoprene units, and examples of tetraterpenes include provitamin A, acyclic lycopene, monocyclic carotene, and bicyclic alpha-carotene, and beta-carotene. Terpenes can also be used as permeability enhancers, effective to enhance the permeability of membranes or tissue to emulsifiers.

D-limonene and its derivatives, such as S-perillic acid and S-perillyl alcohol, comprise terpene emulsifiers of the present invention. It is known in the art that these compounds are quite safe and non-toxic for subjects. Therefore, terpene emulsifiers are contemplated to be effective in treating obesity.

### Examples of Serum and Systemic Circulation Concentrations of Terpene Emulsifiers

Serum and systemic circulation concentrations of a terpene emulsifier effective to treat obesity may vary depending on a number of factors. Influential variables can include, for example, various chemical properties of one terpene, as compared to another. For example different terpenes can differ in pKₐ, solubility, molecular weight, etc., and these properties of a particular terpene may affect how a patient metabolizes the terpene, how much of the terpene enters and remains in the systemic circulation of a subject, and how effectively the terpene treat obesity.

Accordingly, in some embodiments of the present invention, a serum or a systemic circulation concentration of a terpene effective to treat obesity may be in a range of from, for instance, about 1 μM to about 10 μM, about 1 μM to about 20 μM, about 2 μM to about 30 μM, about 3 μM to about 40 μM, about 4 μM to about 50 μM, about 50 μM to about 60 μM, about 60 μM to about 100 μM, about 10 μM to about 125 μM, about 125 μM to about 150 μM, about 150 μM to about 1 μM, about 15 μM to about 175 μM, about 175 μM to about 200 μM, about 20 μM to about 225 μM, about 225 μM to about 250 μM, about 250 to 275 μM, about 275 μM to about 300 μM, about 300 μM to about 325 μM, about 325 μM to about 350 μM, about 350 μM to about 375 μM, about 375 μM to about 400 μM, about 40 μM to about 425 μM, about 425 μM to about 450 μM, about 45 μM to about 475 μM, about 475 μM to about 500 μM, about 500 μM to about 550 μM, about 550 μM to about 600 μM, about 600 μM to about 650 μM, about 650 μM to about 700 μM, about 700 μM to about 750 μM, about 750 μM to about 800 μM, about 800 μM to about 850 μM, about 850 μM to about 900 μM, about 900 μM to about 950 μM, 950 μM to about 1.0 mM, about 1 mM to about 10 mM, about 10 mM to about 20 mM, about 20 mM to about 30 mM, about 30 mM to about 40 mM, about 40 mM to about 50 mM, about 50 mM to about 60 mM, about 60 mM to about 100 mM, about 100 mM to about 125 mM, about 125 mM to about 150 mM, about 150 μM to about 1M, about 150 μM to about 1 mM, about 150 mM to about 175 mM, about 175 mM to about 200 mM, about 200 mM to about 225 mM, about 225 mM to about 250 mM, about 250 mM to about 275 mM, about 275 mM to about 300 mM, about 300 mM to about 325 mM, about 325 mM to about 350 mM, about 350 mM to about 375 mM, about 375 mM to about 400 mM, about 400 mM to about 425 mM, about 425 mM to about 450 mM, about 450 mM to about 475 mM, about 475 mM to about 500 mM, about 500 mM to about 550 mM, about 550 mM to about 600 mM, about 600 mM to about 650 mM, about 650 mM to about 700 mM, about 700 mM to about 750 mM, about 750 mM to about 800 mM, about 800 mM to about 850 mM, about 850 to about 900 mM, about 900 to about 950 mM, about 950 mM to about 1.0 M.

### Examples of Terpene Doses

In some embodiments, a terpene dose effective to treat obesity may be, in weight of administered terpene per kilogram of subject body weight per day (mg/kg/day), in a range of from, for instance, about 1 mg/kg/day to about 10 mg/kg/day, about 10 mg/kg/day to about 20 mg/kg/day, about 20 mg/kg/day to about 30 mg/kg/day, about 30 mg/kg/day to about 40 mg/kg/day, about 40 mg/kg/day to about 50 mg/kg/day, about 50 mg/kg/day to about 60 mg/kg/day, about 60 mg/kg/day to about 100 mg/kg/day, about 100 mg/kg/day to about 125 mg/kg/day, about 125 mg/kg/day to about 150 mg/kg/day, about 150 mg/kg/day to about 175 mg/kg/day, about 175 mg/kg/day to about 200 mg/kg/day, about 200 mg/kg/day to about 225 mg/kg/day, about 225 mg/kg/day to about 250 mg/kg/day, about 250 mg/kg/day to about 275 mg/kg/day, about 275 mg/kg/day to about 300 mg/kg/day, about 300 mg/kg/day to about 325 mg/kg/day, about 325 mg/kg/day to about 350 mg/kg/day, about 350 mg/kg/day to about 375 mg/kg/day, about 375 mg/kg/day to about 400 mg/kg/day, about 400 mg/kg/day to about 425 mg/kg/day, about 425 mg/kg/day to about 450 mg/kg/day, about 450 mg/kg/day to about 475 mg/kg/day, about 475 mg/kg/day to about 500 mg/kg/day, about 500 mg/kg/day to about 550 mg/kg/day, about 550 mg/kg/day to about 600 mg/kg/day, about 600 mg/kg/day to about 650 mg/kg/day, about 650 mg/kg/day to about 700 mg/kg/day, about 700 mg/kg/day to about 750 mg/kg/day, about 750 mg/kg/day to about 800 mg/kg/day, about 800 mg/kg/day to about 850 mg/kg/day, about 850 mg/kg/day to about 900 mg/kg/day, about 900 mg/kg/day to about 950 mg/kg/day, about 950 mg/kg/day to about 1 g/kg/day, about 1 g/kg/day to about 1.25 g/kg/day, about 1.25 g/kg/day to about 1.5 g/kg/day, about 1.5 g/kg/day to about 1.75 g/kg/day, about 1.75 g/kg/day to about 2 g/kg/day, about 2 g/kg/day to about 2.25 g/kg/day, about 2.25 g/kg/day to about 2.5 g/kg/day, about 2.5 g/kg/day to about 2.75 g/kg/day, about 2.750 g/kg/day to about 3 g/kg/day, about 3 g/kg/day to about 4 g/kg/day, about 4 g/kg/day to about 5 g/kg/day, about 5 g/kg/day to about 6 g/kg/day, about 6 g/kg/day to about 7 g/kg/day, about 7 g/kg/day to about 8 g/kg/day, about 8 g/kg/day to about 9 g/kg/day, about 9 g/kg/day to about 10 g/kg/day, about and 10 g/kg/day to about 20 g/kg/day.

### Examples of Saponin Emulsifiers

Saponins are naturally occurring compounds predominantly derived from plants, and can have detergent properties. The name saponin is derived from the soapwort plant (Saponaria) traditionally used in making a type of soap. Saponins are the glycosides of 27 carbon steroids or 30 carbon triterpenes. Removal of the sugar moiety from a saponin by hydrolysis yields the aglycone, sapogenin. Triterpenoid saponins are generally acid, and steroid saponins are generally neutral.

Steroid saponins include three classes of compounds, the cholestanol, furostanol, and spirostanol saponins Examples of furostanol saponins can include, proto-isoeruboside-B and isoeruboside-B, as well as saponins derived, for example, from Ruscus aculeatus, Tacca chantrieri, Solanum hispidum, Dioscorea polygonoides, Tribulus terrestris, and Lilium candidum. Other steroid saponins can include those derived from Saponaria officinalis, Yucca schidigera, and Chlorogalum pomeridianum.

Examples of triterpenoid saponins can include those of the fusidane-lanostante group, cyclopassiflosides, cycloglobiseposides, cycloartanes, dammaranes (e.g., bacopasaponin and jujubogenin), lupanes (e.g., quadranosides), oleananes (e.g., maesapinin), ligatosides, sandrosaponins, pedunsaponins), vulgarsaponin, peduncularisaponin, petersaponin, araliasaponin, assamsaponin, eupteleasaponin, herniariasaponin, jeosaponin, meliltussaponin, ursanes (e.g., randisaponins), brevicuspisaponin, ursolic acid, and indicasaponin. Triterpenoids can also be derived from Quillaja saponaria, as well as those derived from grapes.

Saponins have been identified in plants and animals including, for example, and without being limiting, agave, Agrostemma Githago, alfalfa, aloe, Alfombrilla, Anadenanthera peregrine, amaranth, Angelica sinesis, Aralia chinesis, Aralia manshurica, asparagus, Astragalus membranaceus, buckeyes soapwart, Bacopa monnieri, broomweed, Boussingaultia sp., Bupleurum chinense, Calendula officinalis, Capsicum sp., Christmas Rose, chickweed, chickpeas, Chlorophytum sp., Chlorogalum sp., corn cockle, Codonopsis pilosula, horse chestnuts, curcurbit, Daisies, Dioscorea sp, Drymaria arenaroides, Digitalis sp., Echinodermata, Elecampane, Elutherococcus senticosus, fenugreek, goldenrod, gotu kola, grape skin, Glycyrrhiza glabra, Gymnema sylvestre, Gymnostemma Pentaphyllum, Gypsophila sp., hawthorn, Helleborus niger, Honeylocust, jiaogulan, licorice, lungwort, mullein, Medicago sativa, Cicer arietinum olives, onion, pannax (Koren Ginseng), Platycodon sp, Platycodon grandiflorum, Polygala tenuifola, Quillaja saponaria, quinoa, Phytolacca americana, rambutan, Salvia sp., soapberry, Saponaria sp., Schizandra chinensis, shallots, southern pea, soybean, Tribulus terrestris, Tuberous cucurbit species, Vitis Vinifera, wild yam, yucca, and Zizyphus jujube.

Grapes skin cuticular wax contains saponins. The saponins discovered in the wines contain ursolic acid, oleanolic acid, ursolic aldehyde, oleanolic aldehyde, hydroxyhopanone, damarenolic acid, mastidienonic acid isomasticadienonic acid. The Vitis Vinifera saponins can be used alone or in association with phenolic compounds such as resveratrol.

### Examples of Serum and Systemic Circulation Concentrations of Saponin Emulsifiers

Serum and systemic circulation concentrations of a saponin effective to treat obesity may vary depending on a number of factors. Influential variables can include, for example, various chemical properties of one saponin, as compared to another. For example different saponins can differ in pKₐ, solubility, molecular weight, etc., and these properties of a particular saponin may affect how a patient metabolizes the saponin, how much of the saponin enters and remains in the systemic circulation of a subject, and how effectively the saponin treats obesity.

Accordingly, in some embodiments of the present invention, a serum or a systemic circulation concentration of a saponin effective to treat obesity may be in a range of from, for instance, about 1 μM to about 10 μM, about 5 μM to about 10 μM, about 10 μM to about 20 μM, about 20 μM to about 30 μM, about 30 μM to about 40 μM, about 40 about μM to about 50 μM, about 50 μM to about 60 μM, about 60 μM to about 70 μM, about 70 μM to about 80 μM, about 80 μM to about 90 μM, about 90 μM to about 100 μM, about 50 μM to about 600 μM, about 50 μM to about 100 μM, about 100 μM to about 300 μM, about 100 μM to about 550 μM, about 150 μM to about 1M, about 150 μM to about 1 mM, about 150 μM to about 500 μM, about 200 μM to about 450 μM, about 250 μM to about 400 μM, about 300 μM to about 350 μM, about 500 μM to about 600 μM, about 600 μM to about 700 μM, about 700 μM to about 800 μM, about 800 μM to about 900 μM, about 900 μM to about 1 mM, about 1 mM to about 100 mM, about 100 mM to about 200 mM, about 200 mM to about 300 mM, about 300 mM to about 400 mM, about 400 mM to about 500 mM, about 500 mM to about 600 mM, about 600 mM to about 700 mM, about 700 mM to about 800 mM, about 800 mM to about 900 mM, and about 900 mM to about 1 M.

### Examples of Saponin Doses

In some embodiments, a saponin dose effective to treat obesity may be, in weight of administered saponin per kilogram of subject body weight per day (mg/kg/day), in a range of from, for instance, about 1 mg/kg/day to about 10 mg/kg/day, about 10 mg/kg/day to about 20 mg/kg/day, about 20 mg/kg/day to about 30 mg/kg/day, about 30 mg/kg/day to about 40 mg/kg/day, about 40 mg/kg/day to about 50 mg/kg/day, about 50 mg/kg/day to about 60 mg/kg/day, about 60 mg/kg/day to about 100 mg/kg/day, about 100 mg/kg/day to about 125 mg/kg/day, about 125 mg/kg/day to about 150 mg/kg/day, about 150 mg/kg/day to about 175 mg/kg/day, about 175 mg/kg/day to about 200 mg/kg/day, about 200 mg/kg/day to about 225 mg/kg/day, about 225 mg/kg/day to about 250 mg/kg/day, about 250 mg/kg/day to about 275 mg/kg/day, about 275 mg/kg/day to about 300 mg/kg/day, about 300 mg/kg/day to about 325 mg/kg/day, about 325 mg/kg/day to about 350 mg/kg/day, about 350 mg/kg/day to about 375 mg/kg/day, about 375 mg/kg/day to about 400 mg/kg/day, about 400 mg/kg/day to about 425 mg/kg/day, about 425 mg/kg/day to about 450 mg/kg/day, about 450 mg/kg/day to about 475 mg/kg/day, about 475 mg/kg/day to about 500 mg/kg/day, about 500 mg/kg/day to about 550 mg/kg/day, about 550 mg/kg/day to about 600 mg/kg/day, about 600 mg/kg/day to about 650 mg/kg/day, about 650 mg/kg/day to about 700 mg/kg/day, about 700 mg/kg/day to about 750 mg/kg/day, about 750 mg/kg/day to about 800 mg/kg/day, about 800 mg/kg/day to about 850 mg/kg/day, about 850 mg/kg/day to about 900 mg/kg/day, about 900 mg/kg/day to about 950 mg/kg/day, about 950 mg/kg/day to about 1 g/kg/day, about 1 g/kg/day to about 1.25 g/kg/day, about 1.25 g/kg/day to about 1.5 g/kg/day, about 1.5 g/kg/day to about 1.75 g/kg/day, about 1.75 g/kg/day to about 2 g/kg/day, about 2 g/kg/day to about 2.25 g/kg/day, about 2.25 g/kg/day to about 2.5 g/kg/day, about 2.5 g/kg/day to about 2.75 g/kg/day, about 2.750 g/kg/day to about 3 g/kg/day, about 3 g/kg/day to about 4 g/kg/day, about 4 g/kg/day to about 5 g/kg/day, about 5 g/kg/day to about 6 g/kg/day, about 6 g/kg/day to about 7 g/kg/day, about 7 g/kg/day to about 8 g/kg/day, about 8 g/kg/day to about 9 g/kg/day, about 9 g/kg/day to about 10 g/kg/day, about and 10 g/kg/day to about 20 g/kg/day.

### Examples of Detergent Emulsifiers

Detergents useful as emulsifiers in certain embodiments described herein include ionic detergents, nonionic detergents, and zwitterionic detergents. Detergents can be used to augment or enhance the effectiveness of other emulsifiers, such as bile acids, terpenes, and/or saponins Detergent can also be used as permeability enhancers, effective to enhance the permeability of membranes or tissue to emulsifiers. Exemplary detergents include the following chemical compounds, sometimes characterized by the following tradenames, and their chemical equivalents and their structural derivatives: reduced TRITON® X-100; reduced TRITON® X-114; TRITON® X-100; NP-40; TRITON® X-114; GENAPOL® X-080; GENAPOL® X-100; C12E8; C12E9; THESIT®; LUBROL® PX; GENAPOL® C—100; BRIJ® 35; PLURONIC® F-127®, (laurate); TWEEN® 20 (oleate) and TWEEN® 80; EMPIGEN BB®) (n-dodecyl-N,Ndimethylglycine); ZWITTERGENT® 3-08; ZWITTERGENT® 3-10, ZWITTERGENT® 3-12, ZWITTERGENT® 3-14, ZWITTERGENT® 3-16; CHAPS; CHAPSO; ASB-14; ASB-16; DDMAB; DDMAU; EMPIGEN BB® Detergent; and lauryldimethylamine Oxide (LDAO); BATC Cetyltrimethylammonium Bromide (CTAB); Glycholic Acid, Sodium Salt, TOPPS, Molecular Biology Grade Chenodeoxycholic Acid, sodium salt; Molecular Biology Grade Chenodeoxycholic Acid, Free Acid; APO-10; APO-12; Big CHAP; Big CHAP, deoxy; Cyclo hexyl-n-ethyl-β-D-maltoside; ULTROL® Grade; Cyclo hexyl-n-hexyl-β-D-maltoside, ULTROL® Grade; Cyclohexyl-n-methyl-β-D-maltoside, ULTROL® Grade; n-Decanoylsucrose; n-Decyl-β-D-maltopyranoside, ULTROL® Grade 252718; n-Decyl-β-D-thiomaltoside, ULTROL® Grade; lauroylsarcosine, Sodium Salt n-Dodecyl Sulfate (SDS); SDS, High Purity; SDS, Molecular Biology Grade; SDS; BRIJ® 35, PROTEIN GRADE® Detergent; C12E6 ELUGENT™ Detergent; GENAPOL® C-100, PROTEIN GRADE® Detergent; GENAPOL® X-80, PROTEIN GRADE® Detergent; GENAPOL® X-100, PROTEIN GRADE® Detergent; n-Heptyl-β-D-glucopyranoside; n-Heptyl-β-D-thioglucopyranoside, ULTROL® Grade; n-Hexyl-β-D-glucopyranoside; n-dodecyl-β-D-glucopyranoside 324355; n-Dodecanoylsucrose 324374; Digitonin; Digitonin, alcohol soluble; MEGA-8, ULTROL® Grade, MEGA-9 ULTROL® Grade, MEGA-10 ULTROL® Grade; n-Nonyl-β-D-glucopyranoside; NP-40, PROTEIN GRADE® Detergent; n-Octanoyl-β-D-glucosylamine (NOGA); n-Octanoylsucrose; n-Octyl-β-D-glucopyranoside; n-Octyl-β-D-glucopyranoside, ULTROL® Grade; n-Octyl-β-D-maltopyranoside; n-Octyl-β-D-thioglycopyranoside, ULTROL® Grade; PLURONIC® F-127, PROTEIN GRADE® Detergent; TRITON® X-100, PROTEIN GRADE® Detergent; TRITON® X-100, Molecular Biology Grade; TRITON® X-100, Hydrogenated; TRITON® X-114, PROTEIN GRADE® Detergent; TWEEN® 20; TWEEN® 20, Molecular Biology Grade Detergent; TWEEN® 20, PROTEIN GRADE® Detergent; TWEEN® 80, PROTEIN GRADE® Detergent; n-Undecyl-B-D-maltoside, ULTROL® Grade Detergent; and lauryldimethylamine oxide.

### Examples of Serum and Systemic Circulation Concentrations of Detergents

Serum and Systemic circulation concentrations of a detergent effective to treat obesity may vary depending on a number of factors. Influential variables can include, for example, various chemical properties of one detergent, as compared to another. For example different detergents can differ in pKₐ, solubility, molecular weight, etc., and these properties of a particular detergent may affect how a patient metabolizes the detergent, how much of the detergent enters and remains in the systemic circulation of a subject, and how effectively the detergent treats obesity.

Accordingly, in some embodiments of the present invention, a serum or a systemic circulation concentration of a detergent effective to treat obesity may be in a range of from, for instance, about 1 μM to about 10 μM, about 5 μM to about 10 μM, about 10 μM to about 20 μM, about 20 μM to about 30 μM, about 30 μM to about 40 μM, about 40 about μM to about 50 μM, about 50 μM to about 60 μM, about 60 μM to about 70 μM, about 70 μM to about 80 μM, about 80 μM to about 90 μM, about 90 μM to about 100 μM, about 50 μM to about 600 μM, about 50 μM to about 100 μM, about 100 μM to about 300 μM, about 100 μM to about 550 μM, about 150 μM to about 1M, about 150 μM to about 500 μM, about 200 μM to about 450 μM, about 250 μM to about 400 μM, about 300 μM to about 350 μM, about 500 μM to about 600 μM, about 600 μM to about 700 μM, about 700 μM to about 800 μM, about 800 μM to about 900 μM, about 900 μM to about 1 mM, about 1 mM to about 100 mM, about 100 mM to about 200 mM, about 200 mM to about 300 mM, about 300 mM to about 400 mM, about 400 mM to about 500 mM, about 500 mM to about 600 mM, about 600 mM to about 700 mM, about 700 mM to about 800 mM, about 800 mM to about 900 mM, and about 900 mM to about 1M.

### Examples of Detergent Doses

In some embodiments, a detergent dose effective to treat obesity may be, in weight of administered detergent per kilogram of subject body weight per day (mg/kg/day), in a range of from, for instance, about 1 mg/kg/day to about 10 mg/kg/day, about 10 mg/kg/day to about 20 mg/kg/day, about 20 mg/kg/day to about 30 mg/kg/day, about 30 mg/kg/day to about 40 mg/kg/day, about 40 mg/kg/day to about 50 mg/kg/day, about 50 mg/kg/day to about 60 mg/kg/day, about 60 mg/kg/day to about 100 mg/kg/day, about 100 mg/kg/day to about 125 mg/kg/day, about 125 mg/kg/day to about 150 mg/kg/day, about 150 mg/kg/day to about 175 mg/kg/day, about 175 mg/kg/day to about 200 mg/kg/day, about 200 mg/kg/day to about 225 mg/kg/day, about 225 mg/kg/day to about 250 mg/kg/day, about 250 mg/kg/day to about 275 mg/kg/day, about 275 mg/kg/day to about 300 mg/kg/day, about 300 mg/kg/day to about 325 mg/kg/day, about 325 mg/kg/day to about 350 mg/kg/day, about 350 mg/kg/day to about 375 mg/kg/day, about 375 mg/kg/day to about 400 mg/kg/day, about 400 mg/kg/day to about 425 mg/kg/day, about 425 mg/kg/day to about 450 mg/kg/day, about 450 mg/kg/day to about 475 mg/kg/day, about 475 mg/kg/day to about 500 mg/kg/day, about 500 mg/kg/day to about 550 mg/kg/day, about 550 mg/kg/day to about 600 mg/kg/day, about 600 mg/kg/day to about 650 mg/kg/day, about 650 mg/kg/day to about 700 mg/kg/day, about 700 mg/kg/day to about 750 mg/kg/day, about 750 mg/kg/day to about 800 mg/kg/day, about 800 mg/kg/day to about 850 mg/kg/day, about 850 mg/kg/day to about 900 mg/kg/day, about 900 mg/kg/day to about 950 mg/kg/day, about 950 mg/kg/day to about 1 g/kg/day, about 1 g/kg/day to about 1.25 g/kg/day, about 1.25 g/kg/day to about 1.5 g/kg/day, about 1.5 g/kg/day to about 1.75 g/kg/day, about 1.75 g/kg/day to about 2 g/kg/day, about 2 g/kg/day to about 2.25 g/kg/day, about 2.25 g/kg/day to about 2.5 g/kg/day, about 2.5 g/kg/day to about 2.75 g/kg/day, about 2.750 g/kg/day to about 3 g/kg/day, about 3 g/kg/day to about 4 g/kg/day, about 4 g/kg/day to about 5 g/kg/day, about 5 g/kg/day to about 6 g/kg/day, about 6 g/kg/day to about 7 g/kg/day, about 7 g/kg/day to about 8 g/kg/day, about 8 g/kg/day to about 9 g/kg/day, about 9 g/kg/day to about 10 g/kg/day, about and 10 g/kg/day to about 20 g/kg/day.

### Examples of Pharmaceutical Formulations

Certain embodiments of the present invention provide pharmaceutical formulations comprising biocompatible emulsifiers such as bile acid, terpene, saponin, and/or detergent, and at least one of a sustained release delivery system, an absorption enhancing agent, a liposome, a statin, a blood pressure control agent, a lipase, and a pharmaceutically acceptable buffer.

Sustained Release Delivery Systems

In some embodiments, pharmaceutical formulations of the present invention comprise a sustained release delivery system that results in the maintenance of circulating levels of emulsifiers effective to result in induction of weight loss or prevention of weight gain for extended periods of time, for example, a period of 2 hours or longer. In some embodiments, release is sustained over a period of 24 hours.

In some embodiments, a sustained release delivery system comprises one or more pharmaceutical diluents. Exemplary pharmaceutical diluents include, monosaccharides, disaccharides, polyhydric alcohols, starch, lactose, dextrose, mannitol, sucrose, microcrystalline cellulose, sorbitol, xylitol, fructose, and a combination thereof. In some embodiments, the sustained release delivery system comprises one or more pharmaceutical diluents in an amount of about 5% to about 80% by weight; from about 10% to about 50% by weight; or about 20% by weight of the formulation.

In some embodiments, a sustained release delivery system comprises one or more antiwetting agents, such as a hydrophobic polymer. In certain embodiments, an antiwetting agent is distributed unevenly in the formulation in layers, in pockets, in a coating, or combinations thereof. In certain embodiments, an antiwetting agent is distributed uniformly throughout the formulation. Exemplary hydrophobic polymer antiwetting agents include alkyl celluloses (e.g., C₁₋₆ alkyl celluloses, carboxymethylcellulose), methyl celluloses, ethyl celluloses, propyl celluloses other hydrophobic cellulosic materials or compounds (e.g., cellulose acetate phthalate, hydroxypropylmethylcellulose phthalate), polyvinyl acetate polymers (e.g., polyvinyl acetate phthalate), polymers or copolymers derived from acrylic and/or methacrylic acid esters, zein, waxes (alone or in admixture with fatty alcohols), shellac, hydrogenated vegetable oils, and a combination thereof.

Some embodiments comprise anti-wetting agents in amount of about 0.5% to about 20% by weight of the formulation; in an amount of about 2% to about 10% by weight of the formulation; in an amount of about 3% to about 7% by weight of the formulation; or in an amount of about 5% by weight of the formulation.

In some embodiments, a sustained release delivery system comprises at least one plasticizer, such as triethyl citrate, dibutyl phthalate, propylene glycol, polyethylene glycol, or mixtures of two or more thereof as a coating of the formulation.

In some embodiments, a sustained release delivery system comprises at least one water soluble compound, such as polyvinylpyrrolidone and hydroxypropylmethylcellulose. In certain embodiments, a water soluble compound is distributed unevenly in the formulation in layers, in pockets, as a coating, or combinations thereof. In certain embodiments, a water soluble compound is distributed uniformly throughout the formulation.

In some embodiments, application of a sustained release coating, as described herein, to a formulation may comprise: spraying an aqueous dispersion of the coating onto a core made, for example, by dry or wet granulation of mixed powders of emulsifiers and at least one binding agent; coating an inert bead with emulsifiers and at least one binding agent; and spheronizing mixed powders of emulsifiers and at least one spheronizing agent. Exemplary binding agents include hydroxypropylmethylcelluloses. Exemplary spheronizing agents include microcrystalline celluloses. In some embodiments, the core comprises a tablet made by compressing granules or a powder comprising emulsifiers and/or pharmaceutically acceptable salts or conjugates thereof.

In some embodiments, pharmaceutical formulations comprising emulsifiers and a sustained release delivery system, as described herein, are coated with a sustained release coating, as described herein. In some embodiments, the formulations comprising biocompatible emulsifiers and a sustained release delivery system, as described herein, are coated with a hydrophobic polymer, as described herein. In some embodiments, the formulations comprising emulsifiers and a sustained release delivery system, as described herein, are coated with an enteric coating. Exemplary enteric coatings include cellulose acetate phthalate, hydroxypropylmethylcellulose phthalate, polyvinylacetate phthalate, methacrylic acid copolymer, shellac, hydroxypropylmethylcellulose succinate, cellulose acetate trimelliate, and a combination thereof.

In some embodiments, the pharmaceutical formulations comprising an emulsifier and a sustained release delivery system, as described herein, are coated with a hydrophobic polymer, as described herein, and further coated with an enteric coating. In any of the embodiments described herein, the formulations comprising emulsifiers and a sustained release delivery system, as described herein, can optionally be coated with a hydrophilic coating which can be applied above or beneath a sustained release film, and/or above or beneath the enteric coating.

Absorption Enhancing Agents

In some embodiments, pharmaceutical formulations of the present invention comprise agents that enhance absorption of biocompatible emulsifiers of the invention across, for instance, an intestinal epithelium, a mucosal epithelium, and skin. Absorption enhancing agents include, for example, EDTA, sodium salicylate, sodium caprate, diethyl maleat, N-lauryl-β-D-maltophyranoside, linoleic acid polyoxyethylated, tartaric acid, SDS, Triton X-100, hexylglucoside, hexylmaltoside, heptylglucoside, octylglucoside, octylmaltoside, nonylglucoside, nonylmaltoside, decylglucoside, deceylmaltoside, dodecylmaltoside, tetradecylmaltoside, dodecylglucoside, tridecylmaltoside, as well as mucolytic agents, for example N-acetylcysteine, chitosan, sulfoxides, alcohols, fatty acids and fatty acid esters, polyols, surfactants, terpenes, alkanones, liposomes, ethosomes, cylodextrins, ethanol, glyceryl monoethyl ether, monoglycerides, isopropylmyristate, lauryl alcohol, lauric acid, lauryl lactate, lauryl sulfate, terpinol, menthol, D-limonene, DMSO, polysorbates, N-methylpyrrolidone, polyglycosylated glycerides, Azone®, CPE-215®, NexAct®, SEPA®, and phenyl piperizine. In some embodiments, permeability enhancing agents can also function as emulsifiers.

In some embodiments, biocompatible emulsifiers such as bile acid, terpene, saponin of the present invention also have properties of permeability enhancing agents, as described herein.

In some embodiments, administration of a pharmaceutical formulation across an epithelium results from at least one of iontophoresis, electroporation, sonophoresis, thermal poration, microneedle treatment, and dermabrasion.

In some embodiments, the pharmaceutical formulation is administered so as to achieve circulating levels of at least 50 μM of the emulsifier within 5 minutes after administration. In some embodiments, administration is performed intravenously. In some embodiments, administration occurs intra-arterially. In some embodiments, levels in a range from about 50 μM to about 600 μM are achieved within 5 minutes of administration. In some embodiments, levels in a range from about 100 μM to about 600 μM are achieved within 5 minutes of administration. In some embodiments, levels in a range from about 100 μM to about 300 μM are achieved within 5 minutes of administration.

Liposomes

Some embodiments of the present invention provide pharmaceutical formulations comprising an active ingredient emulsifier or a combination of active ingredient emulsifiers and unilaminar or multilaminer liposomes having an average diameter in a range of from, for instance, about 100 nm to about 200 nm, about 200 nm to about 300 nm, about 300 nm to about 400 nm, about 400 nm to about 500 nm, about 500 nm to about 600 nm, about 600 nm to about 700 nm, about 700 nm to about 800 nm, about 800 nm to about 900 nm, about 900 nm to about 1.0 micrometer, about 1.0 μm to about 1.25 μm, about 1.250 μm to about 1.5 μm, about 1.5 μm to about 1.75 μm, about 1.75 μm to about 2.0 μm, about 2.0 μm to about 2.25 μm, about 2.25 μm to about 2.5 μm, about 2.5 μm to about 2.75 μm, about 2.75 μm to about 3.0 μm, about 3.0 μm to about 3.25 μm, about 3.25 μm to about 3.5 μm, about 3.5 μm to about 3.75 μm, about 3.75 μm to about 4.0 μm, about 4.0 μm to about 4.5 μm, about 4.5 μm to about 5.0 μm, and about 5.0 μm to about 10.0 μm.

In some embodiments, liposomes comprise lipids and/or phospholipids, such as sphingomyelin, distearoyl-phosphatidylethanolamine (DSPE), distearoyl-phosphatidylcholine (DLPC), phosphatidylcholine (PC), phosphatidylethanolamine (PE), and phosphatidylglycerol (PG).

In some embodiments, a liposomal lipids can be modified with a water soluble polymer, such as a polylactic acid polymer, a polyglycolic acid polymer, a polylactic-polyglycolide copolymer, polyethylene glycol (PEG), polyvinylpyrrolidone, polyacrylamide, polyglycerol, and polyaxozline. In some embodiments, a water soluble polymer comprises an average molecular weight in a range of from, for instance, about 0.1 KDa to about 1.0 KDa, about 1.0 KDa to about 5.0 KDa, about 5.0 KDa to about 25 KDa, about 25 KDa to about 50 KDa, about 50 KDa to about 100 KDa, about 100 KDa to about 250 KDa, about 250 KDa to about 500 KDa, and about 500 KDa to about 1000 KDa. In some embodiments, a covalent bond couples a liposomal lipid to a water soluble polymer.

In some embodiments, liposomal lipids comprising a water soluble polymer comprise an amount of the total liposomal lipids in a range of from, for instance, about 1% to about 10%, about 1% to about 5%, about 10% to about 15%, about 15% to about 20%, about 20% to about 25%, about 25% to about 30%, about 30% to about 40%, about 40% to about 50%, about 50% to about 60%, about 60% to about 70%, about 70% to about 80%, about 80% to about 90%, and about 90% to 100% of the total liposomal lipids.

In some embodiments, liposomes are made by packaging liposomal lipid components with at least one biocompatible emulsifier in water, followed by a lyophilization or an extrusion through, for instance, a membrane comprising pores of a selected average size, such as from about 0.05 μm to about 2.0 μm.

In some embodiments, pharmaceutical formulations comprising an active ingredient emulsifier can be percutaneously introduced into the body via percutaneous chemical absorption enhancers such as liposomes, cyclodextrins, and ethosomes. Cyclodextrins comprise a family of cyclic oligosaccharides, composed of 5 or more α-D-glucopyranoside units linked 1−>4, as in amylose (a fragment of starch). To date, the largest well-characterized cyclodextrin contains 32 1,4-anhydroglucopyranoside units, but even at least 150-membered cyclic oligosaccharides are known. Typical cyclodextrins contain a number of glucose monomers ranging from six to eight units in a ring that comprise a cone shape. α-cyclodextrin comprises a six membered sugar ring molecule; β-cyclodextrin comprises a seven membered sugar ring molecule; and γ-cyclodextrin comprises an eight membered sugar ring molecule. In some embodiments, a liposome formulation can be administered in an amount that comprises an amount of cyclodextrin in a range of from, for instance, about 1 mg/day to about 10 mg/day, about 10 mg/day to about 20 mg/day, about 20 mg/day to about 30 mg/day, about 30 mg/day to about 40 mg/day, about 40 mg/day to about 50 mg/day, about 50 mg/day to about 60 mg/day, about 60 mg/day to about 70 mg/day, about 70 mg/day to about 80 mg/day, about 80 mg/day to about 90 mg/day, about 90 mg/day to about 100 mg/day, about 100 mg/day to about 150 mg/day, about 150 mg/day to about 300 mg/day, about 300 mg/day to about 500 mg/day, and about 500 mg/day to about 1000 mg/day.

In some embodiments, pharmaceutical formulations comprising an active ingredient biocompatible emulsifier further comprise ethosomes. Ethosomes comprise ultradeformable vesicles having an aqueous core surrounded by a lipid bilayer. Ethosomes comprise at least one amphiphat (such as phoshatidylcholine), which in aqueous solvents self-assembles into a lipid bilayer that closes into a simple lipid vesicle. By including at least one bilayer softening component (such as a biocompatible surfactant or an amphiphile drug) lipid bilayer flexibility and permeability are greatly increased. The resulting, flexibility and permeability optimized, ethosome can therefore adapt its shape easily and rapidly, by adjusting local concentration of each bilayer component to the local stress experienced by the bilayer. In its basic organization, broadly similar to a liposome, the ethosome differs from more conventional vesicle primarily by its “softer,” more deformable and adjustable membrane. A consequence an ethosome's strong bilayer deformability is the increased ethosome affinity to bind and retain water. An ultradeformable and highly hydrophilic vesicle always tends to avoid dehydration. For example, an ethosome vesicle applied on an open biological surface, such as non-occluded skin, tends to penetrate its barriers and migrate into the water-rich deeper strata to secure hydration. Barrier penetration by ethosomes involves reversible bilayer deformation, without compromising either the vesicle integrity or the barrier properties for the underlying hydration affinity and gradient to remain in place. Being too large to diffuse through the skin, the ethosome needs to find its own route through the organ. The ethosome vesicles use in drug delivery consequently relies on the carrier's ability to widen and overcome the hydrophilic pores in the skin. A concomitant gradual drug agent release from the ethosome allows drug molecules to diffuse and bind to target. Drug transport by an ethosome to an intra-cellular action site may also involve ethosome carrier lipid bilayer fusion with a cell membrane, or active ethosome uptake by the cell by, e.g. endocytosis.

Ethosomes provide for non-invasive delivery of therapeutic molecules across open biological barriers. Ethosome vesicles can transport across mammalian skin, for example, molecules that are too big to diffuse through skin barriers. Other applications include the transport of small molecule drugs which have certain physicochemical properties which would otherwise prevent them from diffusing across a skin barrier. Another characteristic of certain ethosomes is an ability to deliver active drug agents to peripheral, subcutaneous tissue. This ability relies on minimization of the carrier-associated drug clearance through a cutaneous blood vessels plexus in which non-fenestrated blood capillary walls in the skin that, together with the tight junctions between endothelial cells, preclude vesicles getting directly into blood. Ethosome vesicles are prepared in a similar manner as liposomes, except that no separation of the vesicle-associated and free drug is required. Examples include sonicating, extrusion, low shear rates mixing (multilamellar liposomes), or high high-shear homogenizations unilamellar liposomes) of the crude vesicle suspension.

In some embodiments, pharmaceutical formulations comprising an active ingredient emulsifier further comprise ethosomes in a range of weight:weight or weight:volume percentages of from about 1% to about 5%, about 1% to about 10%, about 10% to about 15%, about 15% to about 20%, about 20% to about 25%, about 25% to about 30%, about 30% to about 40%, about 40% to about 50%, about 50% to about 60%, and about 60% to about 70%.

In some embodiments, liposomes are subjected to both lyophilization and extrusion. Some embodiments provide inhalation pharmaceutical formulations comprising liposomes suitable for administration with an inhaler, such as a metered dose inhaler, a dry powder inhaler, and a jet nebulizer. Some embodiments provide pharmaceutical formulations comprising liposomes suitable for administration by injection. Some embodiments provide topical pharmaceutical formulations comprising liposomes, such as creams, lotions, emulsions, pastes, and ointments, which can transdermally deliver a lipo-dissolving compound, such as a bile acid, terpene, saponin, and/or detergent compound. In some embodiments, formulations comprising liposomes include compounds which assist fat metabolism, such as phoshatidylcholine and/or L-carnitine.

In some embodiments, a liposome formulation can be administered in an amount that comprises an amount of liposome in a range of weight:weight or weight:volume percentages of from about 1% to about 5%, about 1% to about 10%, about 10% to about 15%, about 15% to about 20%, about 20% to about 25%, about 25% to about 30%, about 30% to about 40%, about 40% to about 50%, about 50% to about 60%, and about 60% to about 70%.

Statins

In some embodiments, a method of treating overweight or obesity, or at risk of becoming overweight or obese due to, for instance, a family history or lifestyle predisposition toward becoming overweight or obese, comprises treatment with a biocompatible emulsifier as described above, in combination with agents effective to lower cholesterol. For example, a class of compounds known as “statins” are effective to lower cholesterol. Statins are inhibitors of HMG-CoA reductase, the rate limiting enzyme in the synthesis of mevalonate, a key intermediate in the synthesis of cholesterol, from acetyl-CoA.

A variety of natural and synthetic statins are known. These include, for example and without being limiting, atorvastatin, cerivastatin, fluvastatin, lovastatin, mevastatin, pitavastatin, pravastatin, rosuvastatin, and simvastatin. In some embodiments, a method of reducing body weight in a subject comprises treatment with a biocompatible emulsifier as described above in combination with a statin. The statin and emulsifier can be administered concurrently, or sequentially. In some embodiments, the statin and emulsifier can be provided in the same pharmaceutical composition, either as a mixture or in sub-compartments of a single dosage form such as a pill, capsule, injectable, or any other suitable form for administration.

Doses of statins, administered in combination with at least one active ingredient emulsifier of the present invention, effective to treat obesity may vary depending on a number of factors. Influential variables can include, for example, various chemical properties of one statin, as compared to another. For example different statins can differ in pKₐ, solubility, molecular weight, etc., and these properties of a particular statin may affect how a patient metabolizes the statin, how much of the statin enters and remains in the systemic circulation of a subject, and how effectively the statin will be useful for treating obesity.

Accordingly, in some embodiments, a statin dose comprises an amount of statin in a range of from, for instance, about 1 mg/day to about 10 mg/day, about 10 mg/day to about 20 mg/day, about 20 mg/day to about 30 mg/day, about 30 mg/day to about 40 mg/day, about 40 mg/day to about 50 mg/day, about 50 mg/day to about 60 mg/day, about 60 mg/day to about 70 mg/day, about 70 mg/day to about 80 mg/day, about 80 mg/day to about 90 mg/day, about 90 mg/day to about 100 mg/day, about 100 mg/day to about 150 mg/day, about 150 mg/day to about 300 mg/day, about 300 mg/day to about 600 mg/day, and about 500 mg/day to about 1000 mg/day. The statin and emulsifier can be administered concurrently, or sequentially. In some embodiments, the statin and emulsifier can be provided in the same pharmaceutical composition, either as a mixture or in sub-compartments of a single dosage form such as a pill, capsule, injectable, or any other suitable form for administration.

Blood Pressure Control Agents

In some embodiments, biocompatible emulsifiers can be administered in combination with a an agent effective to control blood pressure. For example, in some embodiments emulsifiers are provided simultaneously, or sequentially, with a statin and a compound like amlodipine.

Lipases

Lipases, a subclass of esterases, comprise water-soluble enzymes that catalyze hydrolysis of ester bonds in water-insoluble lipids. Several distinct lipase enzymes are found in nature, and most lipases act at a specific position on the glycerol backbone of a lipid substrate. In addition, most lipases comprise an alpha/beta hydrolase fold and employ a chymotrypsin-like lipid hydrolysis mechanism involving a serine nucleophile, an acid residue (usually aspartic acid), and a histidine. Several lipases hydrolyze circulating fatty acids.

In some embodiments, emulsifiers as described above can be administered in combination with at least one lipase. Exemplary lipases include pancreatic lipase (HPL), hepatic lipase (HL), endothelial lipase, lipoprotein lipase (LPL), lysosomal lipase (LIPA, and also known as acid cholesteryl ester hydrolase), hepatic lipase (LIPC), hormone-sensitive lipase, pancreatic lipase related protein 1 (PLRP1), pancreatic lipase related protein 2 (PLRP2), phospholipases, lipase H (LIPH), lipase I (LIPI), lipase J (LIPJ), lipase K (LIPK), lipase M (LIPM), lipase N (LIPN), monoglyceride lipase (MGLL), diacylglyceride lipase alpha (DAGLA), diacylglyceride lipase beta (DAGLB), and carboxyl ester lipase (CEL).

Doses of lipases, administered in combination with at least one active ingredient emulsifier of the present invention, effective to treat obesity may vary depending on a number of factors. Influential variables can include, for example, various chemical properties of one lipase, as compared to another. For example different lipases can differ in pKₐ, solubility, molecular weight, etc., and these properties of a particular lipase may affect how a patient metabolizes the lipase, how much of the lipase enters and remains in the systemic circulation of a subject, and how effectively the lipase assist in treating obesity.

Accordingly, in some embodiments, a lipase dose comprises an amount of lipase in a range of from, for instance, about 1 mg/day to about 10 mg/day, about 10 mg/day to about 20 mg/day, about 20 mg/day to about 30 mg/day, about 30 mg/day to about 40 mg/day, about 40 mg/day to about 50 mg/day, about 50 mg/day to about 60 mg/day, about 60 mg/day to about 70 mg/day, about 70 mg/day to about 80 mg/day, about 80 mg/day to about 90 mg/day, about 90 mg/day to about 100 mg/day, about 100 mg/day to about 150 mg/day, about 150 mg/day to about 300 mg/day, about 300 mg/day to about 600 mg/day, and about 500 mg/day to about 1000 mg/day. The lipase and emulsifier can be administered concurrently, or sequentially. In some embodiments, the lipase and emulsifier can be provided in the same pharmaceutical composition, either as a mixture or in sub-compartments of a single dosage form such as a pill, capsule, injectable, or any other suitable form for administration.

### Examples of Routes of Administration

Certain embodiments of the present invention comprise routes of administration such as parenteral, transepithelial, transdermal, gavage, oral, sublingual, rectal, vaginal, inhalation, transmucosal, and injection, such as intradermal, subcutaneous, intravenous, and intramuscular injection in dosage unit formulations containing conventional nontoxic pharmaceutically acceptable carriers, adjuvants, and vehicles, as desired. In some embodiments, emulsifiers can be perfused directly into the systemic circulation by way of an implantable pump. Regardless of the route of administration, the dosing of emulsifiers will result in achieving sustained levels of an emulsifier in the systemic circulation effective to treat obesity.

Parenteral administration includes subcutaneous injections, intravenous injections, intramuscular injections, intrasternal injections, and infusion techniques. Parenteral also includes injection into the corpus cavernosum tissue, which can be conducted using any effective injection system including, but not limited to, conventional syringe-and-needle systems or needleless injection devices.

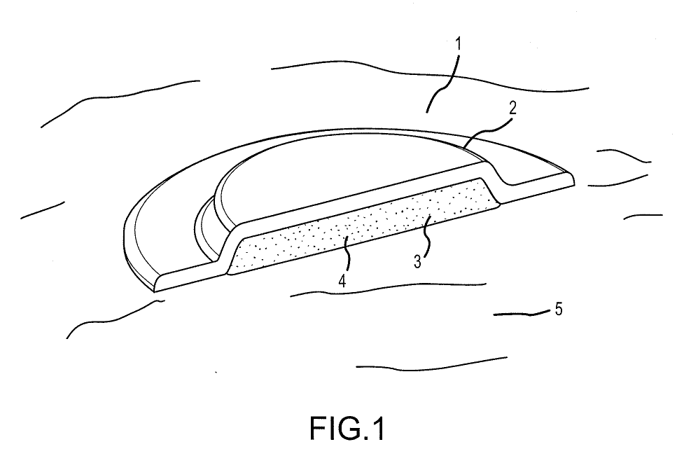

Transdermal drug administration, which is known to one skilled in the art, involves the delivery of pharmaceutical agents via percutaneous passage of the drug into the systemic circulation of the subject. Topical administration, which is well known to one skilled in the art, involves the delivery of pharmaceutical agents via percutaneous passage of the drug into the systemic circulation of the subject. Topical administration includes vaginal administration, vulval administration, penile administration and rectal administration. Topical administration can also involve transdermal patches or iontophoresis devices. Other components can be incorporated into the transdermal patches as well. For example, compositions and/or transdermal patches can be formulated with one or more preservatives or bacteriostatic agents including, but not limited to, methyl hydroxybenzoate, propyl hydroxybenzoate, chlorocresol, benzalkonium chloride, and the like. The skin patch shown in FIG. 1, contains Cholic acid or Chenodeoxycholic acid or Deoxycholic acid or Lithocholic acid or any of their salts or bile salts in general, alone or in combination, or any precursor or derivative of such bile acid or salt, alone or in combination.

For example, the skin patch 1, schematically represented in FIG. 1 is composed of two layers, backing/adhesive layer 2 and reservoir layer 3, filled/impregnated with the biocompatible emulsifier compound 4 above disclosed. Backing/adhesivesubstantially impermeable layer 1 serves the purpose of preventing seeping of emulsifier compound 4 toward the exterior from patch 1 and serves mainly the purpose of permitting adhesion of patch 1 to skin 5. Reservoir layer 2, composed for instance of interwoven fabric impregnated with substance 4, in direct contact with skin 5, serves as reservoir for the delivering of substance 4 thru skin 5 into the systemic circulation.

A skin permeability enhancer along with ordinary excipients can be added to the biocompatible emulsifier(s) in the skin patch to facilitate the penetration and absorption of the ingredients thru the skin. The Percutaneous Chemical Enhancers which can be added can be classified as: Sulfoxides, Alcohols, Fatty acids, Fatty acid esters, Polyols, Amides Surfactants, Terpene, Alkanones Organic acids, Liposomes, Ethosomes, Cyclodextrins. Preferably, the Percutaneous Chemical Enhancers which can be used are: Ethanol, Glyceryl monoethyl ether, Monoglycerides, Isopropylmyristate, Lauryl alcohol, lauric acid, lauryl lactate, lauryl sulfate, Terpinol, Menthol, D-limonene, Beta-cyclodextrin, DMSO acronym for dimethyl sulfoxide, Polysorbates, Fatty acids e.g. oleic, N-methylpyrrolidone, Polyglycosylated glycerides, 1-Dodecylaza cycloheptan-2-one known as Azone®, Cyclopentadecalactone known as CPE-215®, Alkyl-2-(N,N-disubstituted amino)-alkanoate ester, known as NexAct®, 2-(n-nonyl)-1,3-dioxolane known as SEPA®, or phenyl piperazine and the like.

Dosage forms for topical administration of the biocompatible emulsifiers and formulations of the present invention preferably include creams, sprays, lotions, gels, ointments, emulsions, coatings for condoms, liposomes, foams, and the like. Administration of the cream, spray, lotion, gel, ointment, emulsion, coating, liposome, or foam can be accompanied by the use of an applicator or by transurethral drug delivery using a syringe with or without a needle or penile insert or device, or by clitoral, vulval or vaginal delivery, and is within the skill of the art. Typically a lubricant and/or a local anesthetic for desensitization can also be included in the formulation or provided for use as needed. Lubricants include, for example, K-Y Jelly® (available from Johnson & Johnson) or a lidocaine jelly, such as XYLOCAINE® 2% jelly (available from Astra Pharmaceutical Products).

Solid dosage forms for oral administration can include capsules, tablets, effervescent tablets, chewable tablets, pills, powders, sachets, granules and gels. In such solid dosage forms, the active compounds can be admixed with at least one inert diluent such as sucrose, lactose or starch. Such dosage forms can also comprise, as in normal practice, additional substances other than inert diluents, e.g., lubricating agents such as magnesium stearate. In the case of capsules, tablets, effervescent tablets, and pills, the dosage forms can also comprise buffering agents. Soft gelatin capsules can be prepared to contain a mixture of the active compounds or compositions of the present invention and vegetable oil. Hard gelatin capsules can contain granules of the active compound in combination with a solid, pulverulent carrier such as lactose, saccharose, sorbitol, mannitol, potato starch, corn starch, amylopectin, cellulose derivatives of gelatin. Tablets and pills can be prepared with enteric coatings.

Liquid dosage forms for oral administration can include pharmaceutically acceptable emulsions, solutions, suspensions, syrups, and elixirs containing inert diluents commonly used in the art, such as water. Such compositions can also comprise adjuvants, such as wetting agents, emulsifying and suspending agents, and sweetening, and flavoring.

Suppositories for vaginal or rectal administration of the biocompatible emulsifiers and formulations of the invention can be prepared by mixing the biocompatible emulsifiers and formulations with a suitable nonirritating excipient such as cocoa butter and polyethylene glycols which are solid at room temperature but liquid at body temperature, such that they will melt and release the drug.

Injectable preparations, for example, sterile injectable aqueous or oleaginous suspensions can be formulated according to the known art using suitable dispersing agents, wetting agents and/or suspending agents. The sterile injectable preparation can also be a sterile injectable solution or suspension in a nontoxic parenterally acceptable diluent or solvent, for example, as a solution in 1,3-butanediol. Among the acceptable vehicles and solvents that can be used are water, Ringer's solution, and isotonic sodium chloride solution. Sterile fixed oils are also conventionally used as a solvent or suspending medium.

The compounds and compositions of the present invention will typically be administered in a pharmaceutical composition containing one or more carriers or excipients, i.e., pharmaceutically acceptable organic or inorganic carrier substances suitable for parenteral application which do not deleteriously react with the active compounds. Examples of pharmaceutically acceptable carriers include, for example, water, salt solutions, alcohol, silicone, waxes, petroleum jelly, vegetable oils, polyethylene glycols, propylene glycol, liposomes, sugars, gelatin, lactose, amylose, magnesium stearate, talc, surfactants, silicic acid, viscous paraffin, perfume oil, fatty acid monoglycerides and diglycerides, petroethral fatty acid esters, hydroxymethyl-cellulose, polyvinylpyrrolidone, and the like. The compositions can also include one or more permeation enhancers including, for example, dimethylsulfoxide (DMSO), dimethyl formamide (DMF), N,N-dimethylacetamide (DMA), decylmethylsulfoxide (C10MSO), polyethylene glycol monolaurate (PEGML), glyceral monolaurate, lecithin, 1-substituted azacycloheptan-2-ones, particularly 1-N-dodecylcyclazacylcoheptan-2-ones (available under the trademark Azone™ from Nelson Research & Development Co., Irvine, Calif.), alcohols and the like.

The pharmaceutical preparations can be sterilized and if desired, mixed with auxiliary agents which do not deleteriously react with the active compounds, e.g., lubricants, preservatives, stabilizers, wetting agents, salts for influencing osmotic pressure, buffers, colorings, flavoring and/or aromatic substances, and the like. For parenteral application, particularly suitable vehicles consist of solutions, preferably oily or aqueous solutions, as well as suspensions, emulsions, or implants. Aqueous suspensions may contain substances which increase the viscosity of the suspension and include, for example, sodium carboxymethyl cellulose, sorbitol and/or dextran. Optionally, the suspension may also contain stabilizers.

A mentioned previously, the formulations, if desired, can also contain minor amounts of wetting agents and/or pH buffering agents. The composition can be a liquid solution, suspension, emulsion, tablet, pill, capsule, sustained release formulation, or powder. The composition can be formulated as a suppository, with traditional binders and carriers. Oral formulations can include standard carriers such as pharmaceutical grades of mannitol, lactose, starch, magnesium stearate, sodium saccharine, cellulose, magnesium carbonate, and the like.

The compounds and compositions of the present invention can be formulated as pharmaceutically acceptable salts. Pharmaceutically acceptable salts include, for example, alkali metal salts and addition salts of free acids or free bases. The nature of the salt is not critical, provided that it is pharmaceutically-acceptable. Suitable pharmaceutically-acceptable acid addition salts may be prepared from an inorganic acid or from an organic acid. Examples of such inorganic acids include, but are not limited to, hydrochloric, hydrobromic, hydroiodic, nitric (nitrate salt), nitrous (nitrite salt), carbonic, sulfuric and phosphoric acid and the like. Appropriate organic acids include, but are not limited to, aliphatic, cycloaliphatic, aromatic, heterocyclic, carboxylic and sulfonic classes of organic acids, such as, for example, formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, stearic, algenic, O-hydroxybutyric, cyclohexylaminosulfonic, galactaric and galacturonic acid and the like. Suitable pharmaceutically-acceptable base addition salts include, but are not limited to, metallic salts made from aluminum, calcium, lithium, magnesium, potassium, sodium and zinc or organic salts made from primary, secondary and tertiary amines, cyclic amines, N,N′-dibenzylethylenediamine, chloroprocaine, choline, diethanolamine, ethylenediamine, meglumine (N-methylglucamine) and procaine and the like. All of these salts may be prepared by conventional means from the corresponding compound by reacting, for example, the appropriate acid or base with the compound.

### EXPERIMENTAL EXAMPLES

### Protocol 1

Protocol 1 provides an in vivo assay for determining the effectiveness of a biocompatible emulsifier such as a bile salt, bile acid, terpene, saponin, and/or detergent, or a pharmaceutical formulation comprising such a biocompatible emulsifier or combination of emulsifiers, in treating obesity.

In weeks 1 to 8 of protocol 3, four groups of substantially genetically identical mice, Groups A, B, C, and D, each comprising four to twelve animals, are housed in humidity and temperature controlled conditions and fed a high fat and/or high cholesterol rodent chow, such as Picolab Rodent Chow 20 (5053) pellets containing 0.5% (w/w) cholesterol, to promote obesity.

Starting at week 9, Group A mice are fed the high fat and/or high cholesterol rodent chow supplemented with a first emulsifier, such as the bile acid hyodeoxycholic acid (HDCA); Group B mice are fed the high fat and/or high cholesterol rodent chow supplemented with a second emulsifier, such as the terpene emulsifier D-limonene or metabolite thereof; Group C mice are fed the high fat and/or high cholesterol rodent chow supplemented with a combination of the first and second emulsifiers; and Group D mice are fed the high fat and/or high cholesterol rodent chow with no biocompatible emulsifiers (as placebo). Alternatively, Group A mice are fed the high fat and/or high cholesterol rodent chow and administered the first emulsifier by injection, suppository, topical formulation, etc.; Group B mice are fed the high fat and/or high cholesterol rodent chow and administered the second emulsifier by injection, suppository, topical formulation, etc.; Group C mice are fed the high fat and/or high cholesterol rodent chow and administered the combination of the first and second emulsifiers by injection, suppository, topical formulation, etc. The doses of first and second emulsifiers administered to the animals are as described herein.

Starting at week 10, systemic circulation levels of cholesterol and the first and second emulsifiers and/or their precursors, derivatives, metabolites, etc., such as HDCA and D-limonen or S-perillic acid in the systemic circulations of the mice and the levels of ketone bodies excreted via urine can be measured in mice of Groups A, B, C, and D. Assays for determining levels of emulsifiers in blood are known in the art, and include, without limitation, ELISA, high performance liquid chromatography, fast protein liquid chromatography, gas chromatography, thin layer paper chromatography, mass spectrometry and Ketostix®. Assays for determining levels of body fat are also known in the art (e.g., body weight, BMI or body fat percent), which were described above.

At the end of week 25, a comparison of body weight (or BMI) measurements between the animals of Groups A, B, C, and D provides a measure of effectiveness of the tested emulsifier(s) in treating obesity.

### Experiment 1

### The Effects Of Biocompatible Emulsifiers On Fat Metabolism In Mice

In a preliminary in vivo experiment, four groups of mice (each group including three or four mice of same genetic background) were tested for levels of ketone bodies in their urine after consumption of the high fat and/or high cholesterol rodent chow diet supplemented with or without at least on biocompatible emulsifier. In this study, all mice in all groups (all being eight weeks old when this study commenced) were fed with high fat/high cholesterol chow diet (Catalog No. 12079B from Research Diets) for eight weeks. Starting at week 9, the diet of all mice was switched to low fat chow (AIN-76A Rodent Diet from Research Diets). For 15 weeks thereafter, the control mice of group A were fed with pure low fat chow while the mice in treated groups, B, C and D, were fed with low fat chow supplemented with at least one biocompatible emulsifier as follow: Group B mice were fed with low fat chow supplemented with 0.5% Hyodeoxycholic acid (HDCA) and 1.25% D-Limonene (DLM). Group C mice were fed with low fat chow supplemented with 5% DLM. Group D mice were fed with low fat chow supplemented with 1.25% HDCA.

At week 23 of the experiment, urine samples from each mice were taken and analyzed for ketone body levels using Ketostix® stips. The results are listed in the Table below.

TABLE 1
Determination of Ketonuria in Mice

| | | | | Average |
|---|---|---|---|---|
| | | | Ketone | Ketone |
| Mice Group | Feed | Mice ID | level | levels |
| A | Low fat Chow (LFC) | 202R | — | 9 |
| (Control) | only | 203L | — | |
| | | 204R | T | |
| | | 207B | — | |
| B | LFC + 0.5% | 208N | S | 105 |
| | HDCA + | 210R | T | |
| | 1.25% DLM | 211b | T | |
| C | LFC + | 212N | — | 42 |
| | 5% DLM | 213B | T | |
| | | 214L | S | |
| | | 216N | T | |
| D | LFC + | 217B | — | 100 |
| | 1.25% HDCA | 218L | T | |
| | | 220 | S | |

— = No detectable levels of Ketone in the urine
T = Trace amount of Ketone (6 mg/dL) in the urine
S = Small amount of Ketone (15 mg/dL) in the urine

Although preliminary, the results indicates that inclusion of one or more biocompatible emulsifier(s) in the mice diet result in ketonuria in mice. The result suggests that biocompatible emulsifiers in the systemic circulation are capable of inducing fat degradation and, therefore, weight loss.

### Experiment 2

### The Effects Of Biocompatible Emulsifiers On Fat Metabolism In Human

An in vivo experiment was performed to assess the effectiveness of HDCA in causing fat catabolism and treating obesity in human. In this experiment, an overweight individual otherwise healthy took 250 mg of HDCA three times a day (750 mg/day) for a period of 10 weeks without caloric restriction. Weekly testing of this individual for ketonuria using Ketostix® stips showed a mild ketotic state. This experiment demonstrates that biocompatible emulsifiers (in this case HDCA) in systemic circulation may be associated with enhanced ketone production with possible implications for body fat loss and weight loss

The skilled artisan will recognize the interchangeability of various features from different embodiments. Similarly, the various features and steps discussed above, as well as other known equivalents for each such feature or step, can be mixed and matched by one of ordinary skill in this art to perform compositions or methods in accordance with principles described herein. Although the disclosure has been provided in the context of certain embodiments and examples, it will be understood by those skilled in the art that the disclosure extends beyond the specifically described embodiments to other alternative embodiments and/or uses and obvious modifications and equivalents thereof. Accordingly, the disclosure is not intended to be limited by the specific disclosures of embodiments herein.

What is claimed is:

1. A pharmaceutical formulation for treating obesity in a subject, comprising a hyodeoxycholic acid (HDCA) or a pharmaceutically acceptable salt, conjugate, hydrate, solvate, polymorph, or mixture thereof; wherein the HDCA or pharmaceutically acceptable salt, conjugate, hydrate, solvate, polymorph, or mixture thereof is in an amount effective to reduce the subject's body weight by at least 5% within three months after an onset of administration.

2. The pharmaceutical formulation of claim 1, wherein the HDCA or pharmaceutically acceptable salt, conjugate, hydrate, solvate, polymorph, or mixture thereof is in an amount effective to result in a serum concentration in the subject of greater than 50 μM, sustained over at least three months.

3. The pharmaceutical formulation of claim 1, wherein the HDCA or pharmaceutically acceptable salt, conjugate, hydrate, solvate, polymorph, or mixture thereof is in an amount effective to result in a serum concentration in the subject of from about 150 μM to about 1 M.

4. The pharmaceutical formulation of claim 1, wherein the formulation comprises a sustained release formulation.

5. A method of treating obesity in a subject, comprising administering to the subject a pharmaceutical formulation comprising hyodeoxycholic acid (HDCA) or a pharmaceutically acceptable salt, conjugate, hydrate, solvate, polymorph, or mixture thereof in an amount effective to reduce the subject's body weight by at least 5% within three months after an onset of administration.

6. A method of treating obesity, comprising administering to a subject a pharmaceutical formulation comprising hyodeoxycholic acid (HDCA) or a pharmaceutically acceptable salt, conjugate, hydrate, solvate, polymorph, or mixture thereof in an amount effective to induce in a first patient population a mean weight loss that exceeds, by at least 5%, a mean weight loss observed in second patient population to whom a placebo was administered.

7. A method of treating obesity in a subject, comprising administering to the subject a pharmaceutical formulation comprising hyodeoxycholic acid (HDCA) or a pharmaceutically acceptable salt, conjugate, hydrate, solvate, polymorph, or mixture thereof in an amount effective to result in a proportion of subjects receiving the formulation who reach and maintain for at least one year a loss of at least 5% of their body weight to be significantly greater than a proportion of subjects receiving a placebo who reach and maintain for at least one year a loss of at least 5% of their body weight.

8. The method of claim 5, wherein the HDCA is in an amount effective to result in a serum concentration in the subject of greater than 50 μM.

9. The method of claim 5, wherein the HDCA is in an amount effective to result in a serum concentration in the subject of from 150 μM to 1 M.

10. The method of claim 5, wherein the HDCA is in an amount effective to result in a serum concentration in the subject of from 150 μM to 1 mM.

11. The method of claim 5, wherein the formulation comprises a sustained release formulation.

12. The method of claim 5, wherein, during the administering, the subject consumes a diet restricted in carbohydrates to less than 40 grams per day.

13. The method of claim 5, wherein, during the administering, the subject consumes a diet restricted in carbohydrates to less than 60 grams per day.

14. The method of any claim 5, wherein, during the administering, the subject consumes a diet restricted in carbohydrates to less than 20 grams per day.

15. The method of claim 5, wherein, during the administering, the subject consumes a diet restricted in carbohydrates to less than 40 grams per day.
